(19) United States
(12) Patent Application Publication
(10) Pub. No.: US 2023/0405118 A1
(43) Pub. Date: Dec. 21, 2023

(54) STAT-ACTIVATED MACROPHAGES, COMPOSITIONS, AND USES THEREOF

(71) Applicant: THE GEORGE WASHINGTON UNIVERSITY, A CONGRESSIONALLY CHARTERED NOT-FOR-PROFIT CORPORATION, Washington, DC (US)

(72) Inventors: Alejandro VILLAGRA, Falls Church, VA (US); Satish NOONEPALLE, McLean, VA (US); Maria del Mar Gracia HERNANDEZ, Reston, VA (US)

(73) Assignee: THE GEORGE WASHINGTON UNIVERSITY, A CONGRESSIONALLY CHARTERED NOT-FOR-PROFIT CORPORATION, Washington, DC (US)

(21) Appl. No.: 18/251,060

(22) PCT Filed: Oct. 29, 2021
(86) PCT No.: PCT/US2021/057418

§ 371 Date: Apr. 28, 2023

Related U.S. Application Data
(60) Provisional application No. 63/108,027, filed on Oct. 30, 2020.

(51) Int. Cl. A61K 39/00; C12N 5/0786; A61K 35/15; A61K 45/06

(52) U.S. Cl. CPC A61K 39/4614; C12N 5/0645; A61K 39/464495; A61K 39/464491; A61K 39/464486; A61K 39/464488; A61K 39/46449; A61K 39/464482; A61K 39/464481; A61K 39/46447; A61K 39/464456; A61K 39/464451; A61K 39/4644; A61K 35/15; A61K 45/06; C12N 2501/2304; C12N 2501/2313; A61K 2039/5154

(57) ABSTRACT

The present disclosure provides signal transducer and activator of transcription (STAT)-activated macrophages, compositions comprising STAT-activated macrophages, methods of making STAT-activated macrophages, and methods of treating diseases, e.g., cancer, by administering a therapeutically effective amount of STAT-activated macrophages.

### TECHNICAL FIELD

This disclosure provides signal transducer and activator of transcription (STAT)-activated macrophages, compositions comprising STAT-activated macrophages, methods of making STAT-activated macrophages, and methods of treating diseases, e.g., cancer, by administering a therapeutically effective amount of STAT-activated macrophages or a pharmaceutical composition comprising STAT-activated macrophages.

### BACKGROUND OF THE INVENTION

Macrophages play an important role in host innate and adaptive immune responses. They help maintain tissue homeostasis, repair, and fight infections. Macrophages exhibit functional heterogeneity based on their phenotype. They are classified into “M1” or “classically activated” and “M2” or “alternatively activated” macrophages. M2 macrophages secrete anti-inflammatory cytokines such as TGFβ and IL-10, which are generally associated with tumors and function by promoting tumor growth, angiogenesis, tumor invasion, and migration. On the contrary, M1 macrophages secrete pro-inflammatory cytokines such as IL-12 and TNFα and have an anti-tumor function. M1 macrophages also actively scan the tumor microenvironment (TME) for tumor-associated antigens (TAA) and present them to CD8 T-cells to elicit anti-tumor immunity. Thus, the ratio of M1/M2 macrophages in the TME plays a critical role in the TME.

There exists a need for therapeutic strategies that decrease M2 macrophages or increase M1 macrophages in the TME in order to increase anti-tumor immunity.

### SUMMARY OF THE INVENTION

Applicant has unexpectedly discovered that isolated macrophages are reprogrammed outside the body (ex vivo) and polarized towards the anti-tumor M1 phenotype by treatment with a STAT inhibitor. These STAT-activated macrophages can be administered to a subject to treat cancer and other diseases.

In one aspect, the present disclosure provides STAT-activated macrophages.

In another aspect, the present disclosure provides a composition comprising STAT-activated macrophages.

In another aspect, the present disclosure provides a method of making STAT-activated macrophages, the method comprising isolating naïve macrophages from a subject and treating the isolated naïve macrophages ex vivo with a STAT inhibitor.

In another aspect, the present disclosure provides a method of treating a subject in need thereof comprising administering to the subject a therapeutically effective amount of STAT-activated macrophages or a composition comprising STAT-activated macrophages, wherein the subject has cancer, pulmonary fibrosis, liver fibrosis, or heart fibrosis.

### BRIEF DESCRIPTION OF THE DRAWINGS

FIG. 1 is a graph showing polarization efficiency with CD206 as a marker for M2 macrophages when analyzed by flow cytometry in M0 macrophages, vehicle treated M2 macrophages, and M2 macrophages pre-treated with Nexturastat A (NA) or Stattic.

FIG. 2 is a graph showing the arginase I expression levels of macrophages pre-treated with Nexturastat A (NA) or Stattic.

FIG. 3 is a graph showing the TGFβ expression levels of macrophages pre-treated with Nexturastat A (NA) or Stattic.

FIG. 4 is a graph showing the FIZZ1 expression levels of macrophages pre-treated with Nexturastat A (NA) or Stattic.

### DETAILED DESCRIPTION OF THE INVENTION

### I. Compositions of the Disclosure

In one aspect, the present disclosure provides STAT-activated macrophages.

In another aspect, the present disclosure provides compositions comprising STAT-activated macrophages.

In another aspect, the STAT-activated macrophages are produced from naïve macrophages that have been isolated from a subject and treated ex vivo with a STAT inhibitor. In another aspect, the subject is a mammal. In another aspect, the subject is a human. In another aspect, the naïve macrophages are allogeneic macrophages, autologous macrophages, or a combination of allogeneic macrophages and autologous macrophages. In another aspect, the naïve macrophages are allogeneic macrophages. In another aspect, the naïve macrophages are autologous macrophages.

In another aspect, STAT-activated macrophages are produced from naïve macrophages that have been isolated from a subject and treated ex vivo one time with a STAT inhibitor.

In another aspect, the STAT-activated macrophages are produced from naïve macrophages that have been isolated from a subject and treated ex vivo two or more times with a STAT inhibitor.

In another aspect, the STAT-activated macrophages are produced from naïve macrophages that have been isolated from a subject and treated ex vivo with a STAT inhibitor for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

In another aspect, the STAT-activated macrophages are produced from naïve macrophages that have been isolated from a subject and treated ex vivo with a STAT inhibitor and a macrophage polarizing agent. In another aspect, the macrophage polarizing agent comprises lipopolysaccharide (LPS), interferon-gamma, interleukin-4, or interleukin-13, or a combination thereof. In another aspect, the isolated naïve macrophages are treated with the STAT inhibitor before treatment with the macrophage polarizing agent. In another aspect, the isolated naïve macrophages are treated with the STAT inhibitor after treatment with the macrophage polarizing agent. In another aspect, the isolated naïve macrophages are simultaneously treated with the STAT inhibitor and the macrophage polarizing agent. In another aspect, the ex vivo treatment with the macrophage polarizing agent is for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

In another aspect, the STAT-activated macrophages are produced from naïve macrophages that have been isolated from a subject and treated ex vivo with a STAT inhibitor and a tumor antigen. In another aspect, the antigen comprises alphafetoprotein (AFP), carcinoembryonic antigen (CEA), cancer antigen 125 (CA-125), cancer antigen 15-3 (CA 15-3), cancer antigen 19-9 (CA 19-9), mucin 1 (MUC-1), Epithelial tumor antigen (ETA), tyrosinase, or melanoma-associated antigen (MAGE), B melanoma antigen (BAGE), CAGE, New York esophageal squamous cell carcinoma 1 (NY-ESO-1), melanoma antigen recognized by T cells 1 (MART-1), prostate specific antigen (PSA), human epidermal growth factor receptor 2 (HER2), calcitonin, p53, or a combination thereof. In another aspect, the one or more tumor antigens comprise alphafetoprotein (AFP), carcinoembryonic antigen (CEA), CA-125, MUC-1, epithelial tumor antigen (ETA), tyrosinase, Melanoma-associated antigen (MAGE), or p53, or a combination thereof. In another aspect, the isolated naïve macrophages are treated with the STAT inhibitor before treatment with the tumor antigen. In another aspect, the isolated naïve macrophages are treated with the STAT inhibitor after treatment with the tumor antigen. In another aspect, the isolated naïve macrophages are simultaneously treated with the STAT inhibitor and the tumor antigen. In another aspect, the ex vivo treatment with the tumor antigen is for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

In another aspect, the STAT-activated macrophages are produced from naïve macrophages that have been isolated from a subject and treated ex vivo with a STAT inhibitor and a selective HDAC6 inhibitor. In another aspect, the isolated naïve macrophages are treated with the STAT inhibitor before treatment with the selective HDAC6 inhibitor. In another aspect, the isolated naïve macrophages are treated with the STAT inhibitor after treatment with the selective HDAC6 inhibitor. In another aspect, the isolated naïve macrophages are simultaneously treated with the STAT inhibitor and the selective HDAC6 inhibitor. In another aspect, the ex vivo treatment with selective HDAC6 inhibitor is for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

In another aspect, the STAT-activated macrophages are produced from naïve macrophages that have been isolated from a subject and treated ex vivo with a STAT inhibitor, a macrophage polarizing agent, a tumor antigen, and a selective HDAC6 inhibitor. The naïve macrophages can be treated with the STAT inhibitor, the macrophage polarizing agent, the tumor antigen, and the selective HDAC6 inhibitor simultaneously or separately in any order. For example, the naïve macrophages can be treated ex vivo first with the STAT inhibitor followed by the macrophage polarizing agent followed by the tumor antigen followed by the selective HDAC6 inhibitor; the naïve macrophages can be treated ex vivo first with the macrophage polarizing agent followed by the STAT inhibitor followed by the tumor antigen followed by the selective HDAC6 inhibitor; the naïve macrophages can be treated ex vivo first with the STAT inhibitor followed by the tumor antigen followed by the macrophage polarizing agent followed by the selective HDAC6 inhibitor; the naïve macrophages can be treated ex vivo first with the selective HDAC6 inhibitor followed by the STAT inhibitor followed by the macrophage polarizing agent followed by the tumor antigen; and so on. In another aspect, the ex vivo treatment is a STAT inhibitor, a macrophage polarizing agent, a tumor antigen, and a selective HDAC6 inhibitor, independently for each agent, for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

STAT-activated macrophages may be formulated as pharmaceutical compositions or medicaments for clinical use and may comprise a pharmaceutically acceptable carrier, diluent, excipient or adjuvant. Thus, in one aspect, the present disclosure provides compositions comprising STAT-activated macrophages and a pharmaceutically acceptable carrier, adjuvant, excipient or diluent. Pharmaceutically acceptable carriers, diluents, excipients, or adjuvants are known in the art.

The composition may be formulated for parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion.

Suitable formulations may comprise STAT-activated macrophages in a sterile or isotonic medium, e.g., water for injection (WFI). Medicaments and pharmaceutical compositions may be formulated in fluid, including gel, form. Fluid formulations may be formulated for administration by injection or infusion (e.g. via catheter) to a selected region of the human or animal body.

In one aspect, compositions comprising STAT-activated macrophages are formulated for intratumoral or intravenous administration, e.g., for macrophage-directed cancer immunotherapy. See, e.g., Mills et al., Cancer Research 76:513-516 (2016); Lee et al., J Control Release 240:527-540 (2016).

In accordance with the present disclosure, methods are provided for the production of pharmaceutically useful compositions, such methods of production may comprise one or more steps selected from isolating/purifying STAT-activated macrophages produced according to the methods described herein; and/or mixing STAT-activated macrophages produced according with a pharmaceutically acceptable carrier, adjuvant, excipient or diluent.

For example, one aspect of the present disclosure relates to a method of formulating or producing a medicament or pharmaceutical composition, the method comprising formulating a pharmaceutical composition or medicament by mixing STAT-activated macrophages produced according to the methods described herein with a pharmaceutically acceptable carrier, adjuvant, excipient or diluent.

### II. Methods of Producing STAT-Activated Macrophages

In one aspect, the present disclosure provides methods of producing STAT-activated macrophages, the methods comprising isolating naïve macrophages from a subject and treating the isolated naïve macrophages ex vivo with a STAT inhibitor. In another aspect, the subject is a mammal. In another aspect, the subject is a human. In another aspect, the naïve macrophages are allogeneic macrophages, autologous macrophages, or a combination of allogeneic macrophages and autologous macrophages. In another aspect, the naïve macrophages are allogeneic macrophages. In another aspect, the naïve macrophages are autologous macrophages.

In another aspect, STAT-activated macrophages are produced from naïve macrophages that have been isolated from a subject and treated ex vivo one time with a STAT inhibitor.

In another aspect, the STAT-activated macrophages are produced from naïve macrophages that have been isolated from a subject and treated ex vivo two or more times with a STAT inhibitor.

In another aspect, the STAT-activated macrophages are produced from naïve macrophages that have been isolated from a subject and treated ex vivo with a STAT inhibitor for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

In another aspect, the selective STAT inhibitor is any one or more of the inhibitors provided in Table 1.

TABLE 1

| | | | | |
|---|---|---|---|---|
| S3I-201 | Bosutinib | Fludarabine | WP1066 | Reveratrol |
| | (SKI-606) | | | |
| Kaempferol-3-O- | RCM-1 | Falcarindiol | Cucurbitacin | STAT3-IN-1 |
| rutinoside | | | IIb | |
| STAT5-IN-1 | Stattic | TPCA-1 | Niclosamide | Nifuroxazide |
| SH-4-54 | AS1517499 | Napabucasin | Artesunate | BP-1-102 |
| | | (BBI608) | | |
| Cryptotanshinone | SH5-07 | Homoharringtonine | Ochromycinone | HJC0152 |
| | (SH-5-07) | | (STA-21) | |
| Scutellarin | APTSTAT3- | C188-9 | HO-3867 | OPB-31121 |
| | 9R | | | |
| Corosolic acid | Oleanolic | Chlorogenic acid | SS610 | S3I-M2001 |
| | acid | | | |

In another aspect, the STAT inhibitor is a STAT1 inhibitor, a STAT2 inhibitor, a STAT3 inhibitor, a STAT4 inhibitor, a STAT5A inhibitor, a STAT5B inhibitor, or a STATE inhibitor.

In another aspect, the STAT3 inhibitor is 6-nitrobenzo[b]thiophene-1,1-dioxide (Stattic).

In another aspect, the STAT-activated macrophages are produced from naïve macrophages that have been isolated from a subject and treated ex vivo with a STAT inhibitor and a macrophage polarizing agent. In another aspect, the macrophage polarizing agent comprises lipopolysaccharide (LP S), interferon-gamma, interleukin-4, or interleukin-13, or a combination thereof. In another aspect, the isolated naïve macrophages are treated with the STAT inhibitor before treatment with the macrophage polarizing agent. In another aspect, the isolated naïve macrophages are treated with the STAT inhibitor after treatment with the macrophage polarizing agent. In another aspect, the isolated naïve macrophages are simultaneously treated with the STAT inhibitor and the macrophage polarizing agent. In another aspect, the isolated naïve macrophages are treated with the macrophage polarizing agent for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

In another aspect, the STAT-activated macrophages are produced from naïve macrophages that have been isolated from a subject and treated ex vivo with a STAT inhibitor and a tumor antigen. In another aspect, the antigen comprises alphafetoprotein (AFP), carcinoembryonic antigen (CEA), cancer antigen 125 (CA-125), cancer antigen 15-3 (CA 15-3), cancer antigen 19-9 (CA 19-9), mucin 1 (MUC-1), epithelial tumor antigen (ETA), tyrosinase, or melanoma-associated antigen (MAGE), B melanoma antigen (BAGE), CAGE, New York esophageal squamous cell carcinoma 1 (NY-ESO-1), melanoma antigen recognized by T cells 1 (MART-1), prostate specific antigen (PSA), human epidermal growth factor receptor 2 (HER2), calcitonin, p53, or a combination thereof. In another aspect, the antigen comprises alphafetoprotein (AFP), carcinoembryonic antigen (CEA), CA-125, MUC-1, epithelial tumor antigen (ETA), tyrosinase, Melanoma-associated antigen (MAGE), or p53, or a combination thereof. In another aspect, the isolated naïve macrophages are treated with the STAT inhibitor before treatment with the tumor antigen. In another aspect, the isolated naïve macrophages are treated with the STAT inhibitor after treatment with the tumor antigen. In another aspect, the isolated naïve macrophages are simultaneously treated with the STAT inhibitor and the tumor antigen. In another aspect, the isolated naïve macrophages are treated with the tumor antigen for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

In another aspect, the STAT-activated macrophages are produced from naïve macrophages that have been isolated from a subject and treated ex vivo with a STAT inhibitor and a selective HDAC6 inhibitor. In another aspect, the isolated naïve macrophages are treated with the STAT inhibitor before treatment with the selective HDAC6 inhibitor. In another aspect, the isolated naïve macrophages are treated with the STAT inhibitor after treatment with the selective HDAC6 inhibitor. In another aspect, the isolated naïve macrophages are simultaneously treated with the STAT inhibitor and the selective HDAC6 inhibitor. In another aspect, the isolated naïve macrophages are treated with the selective HDAC6 inhibitor for 72 hours or less, e.g., 48 hours or less, 36 hours or less, 24 hours or less, 12 hours or less, 8 hours or less, 6 hours or less, 5 hours or less, 4 hours or less, 3 hours or less, 2 hours or less, 1 hour or less, or 30 minutes or less.

In another aspect, the selective HDAC6 inhibitor is a compound of Formula I, or a pharmaceutically acceptable salt thereof. See above. In another aspect, wherein R^{6a}, R^{6b}, R^{6c}, R^{6d}, and R^{6e} are independently selected from the group consisting of hydrogen, halogen, hydroxy, nitro, cyano, —NR^{a}R^{b}, —C(═O)NR^{a}R^{b}, —C(═O)R^{c}, C₁₋₄ alkyl, C₁₋₄ alkoxy, and C₁₋₄ haloalkyl. In another aspect, R^{6a}, R^{6b}, R^{6c}, R^{6d}, and R^{6e} are each independently selected from the group consisting of hydrogen, halogen, cyano, C₁₋₄ alkyl, and C₁₋₄ alkoxy. In another aspect n is 1. In another aspect, n is 2. In another aspect, n can be 3.

In another aspect, the selective HDAC6 inhibitor is a compound of Formula II, or a pharmaceutically acceptable salt thereof. See above. In another aspect, R^{7a}, R^{7b}, R^{7c}, R^{7d}, and R^{7e} are each independently selected from the group consisting of hydrogen, halogen, hydroxy, nitro, cyano, —NR^{a}R^{b}, —C(═O)NR^{a}R^{b}, —C(═O)R^{c}, C₁₋₄ alkyl, C₁₋₄ alkoxy, and C₁₋₄ haloalkyl. In another aspect, R^{7a}, R^{7b}, R^{7c}, R^{7d}, and R^{7e} are each independently selected from the group consisting of hydrogen, halogen, cyano, C₁₋₄ alkyl, and C₁₋₄ alkoxy. In another aspect, n is 1. In another aspect, n is 2. In another aspect, n is 3.

In another aspect, the selective HDAC6 inhibitor is a compound of Formula III, or a pharmaceutically acceptable salt thereof. See above. In another aspect, m is 0 and [symbol] is a double bond. In another aspect, m is 1 and [symbol] is a single bond. In another aspect, n is 1. In another aspect, n is 2. In another aspect, n is 3.

In another aspect, the selective HDAC6 inhibitor that is a compound of Formula IV, or a pharmaceutically acceptable salt thereof. See above. In another aspect, n is 1. In another aspect, n is 2. In another aspect, n is 3.

In another aspect, the selective HDAC6 inhibitor is a compound of Table 2, or a pharmaceutically acceptable salt thereof.

In another aspect, the selective HDAC6 inhibitor is at least 20-fold selective over one or more other HDAC isoforms, e.g., HDAC1, HDAC2, HDAC3, HDAC4, HDAC5, HDAC7, HDAC8, HDAC9, HDAC10, or HDAC11.

In another aspect, the selective HDAC6 inhibitor is at least 100-fold selective over one or more other HDAC isoforms.

In another aspect, the selective HDAC6 inhibitor is at least 600-fold selective over one or more other HDAC isoforms.

### III. Treating Disease by Adoptive Transfer

STAT-activated macrophages or pharmaceutical compositions comprising STAT-activated macrophages may be useful for adoptive cell therapy. Adoptive cell therapy involves the introduction of cells into a subject in need of treatment. In some cases, the cells are derived from the subject that they are introduced to (autologous cell therapy). See, e.g., Moroni et al., Nature Medicine 25:1560-1565 (2019). That is, cells, e.g., macrophages, may have been obtained from the patient, activated according to methods described herein, and then returned to the same subject. Methods disclosed herein may also be used in allogenic cell therapy, in which cells obtained from a different individual are introduced into the subject.

In one aspect, the present disclosure provides methods of treating or preventing a disease or disorder a subject in need thereof, the methods comprising administering to the subject a therapeutically effective amount of STAT-activated macrophages or a composition comprising STAT-activated macrophages. In another aspect, the disease or disorder is cancer, pulmonary fibrosis, liver fibrosis, or heart fibrosis.

In another aspect, the present disclosure provides methods of treating or preventing a disease or disorder in a subject in need thereof, the methods comprising:
(a) isolating naïve macrophages from a subject;
(b) treating the naïve macrophages ex vivo with a STAT inhibitor to produce STAT-activated macrophages; and;
(c) administering the STAT-activated macrophages to the subject.

In another aspect, the present disclosure provides methods of treating or preventing a disease or disorder in a subject in need thereof, the methods comprising:
(a) isolating naïve macrophages from a subject;
(b) treating the naïve macrophages ex vivo with a STAT inhibitor to produce STAT-activated macrophages;
(c) treating the STAT-activated macrophages with a macrophage polarizing agent; and
(d) administering the STAT-activated macrophages to the subject.

In another aspect, the present disclosure provides methods of treating or preventing a disease or disorder in a subject in need thereof, the methods comprising:
(a) isolating naïve macrophages from a subject;
(b) treating the naïve macrophages ex vivo with a STAT inhibitor to produce STAT-activated macrophages;
(c) treating the STAT-activated macrophages with a tumor antigen; and
(d) administering the STAT-activated macrophages to the subject.

In another aspect, the present disclosure provides methods of treating or preventing a disease or disorder in a subject in need thereof, the methods comprising:
(a) isolating naïve macrophages from a subject;
(b) treating the naïve macrophages ex vivo with a STAT inhibitor to produce STAT-activated macrophages;
(c) treating the STAT-activated macrophages with a selective HDAC6 inhibitor; and
(d) administering the STAT-activated macrophages to the subject.

In another aspect, the present disclosure provides methods of treating or preventing a disease or disorder in a subject in need thereof, the methods comprising:
(a) isolating naïve macrophages from a subject;
(b) treating the naïve macrophages ex vivo with a STAT inhibitor to produce STAT-activated macrophages;
(c) treating the STAT-activated macrophages with a macrophage polarizing agent;
(d) treating the STAT-activated macrophages with a tumor antigen; and
(e) administering the STAT-activated macrophages to the subject.

In another aspect, the present disclosure provides methods of treating or preventing a disease or disorder in a subject in need thereof, the methods comprising:
(a) isolating naïve macrophages from a subject;
(b) treating the naïve macrophages ex vivo with a STAT inhibitor to produce STAT-activated macrophages;
(c) treating the STAT-activated macrophages with a macrophage polarizing agent;
(d) treating the STAT-activated macrophages with a selective HDAC6 inhibitor; and
(e) administering the STAT-activated macrophages to the subject.

In another aspect, the present disclosure provides methods of treating or preventing a disease or disorder in a subject in need thereof, the methods comprising:
(a) isolating naïve macrophages from a subject;
(b) treating the naïve macrophages ex vivo with a STAT inhibitor to produce STAT-activated macrophages;
(c) treating the STAT-activated macrophages with a tumor antigen;
(d) treating the STAT-activated macrophages with a selective HDAC6 inhibitor; and
(e) administering the STAT-activated macrophages to the subject.

In another aspect, the present disclosure provides methods of treating or preventing a disease or disorder in a subject in need thereof, the methods comprising:
(a) isolating naïve macrophages from a subject;
(b) treating the naïve macrophages ex vivo with a STAT inhibitor to produce STAT-activated macrophages;
(c) treating the STAT-activated macrophages with a macrophage polarizing agent;
(d) treating the STAT-activated macrophages with a tumor antigen;
(e) treating the STAT-activated macrophages with a selective HDAC6 inhibitor; and
(f) administering the STAT-activated macrophages to the subject.

In one aspect, the subject from which the naïve macrophages are isolated is the subject administered with STAT-activated macrophages, i.e., adoptive transfer is of autologous cells. In some aspects, the subject from which the naïve macrophages are isolated is a different subject than the subject to which the STAT-activated macrophages are administered, i.e., adoptive transfer is of allogenic cells.

In one aspect, methods of treating or preventing a disease or disorder in a subject comprise one or more of the following steps: taking a biological sample from the subject; isolating naïve macrophages from the biological sample; treating the naïve macrophages ex vivo with a STAT inhibitor; treating the treated macrophages with a macrophage polarizing agent; treating the treated macrophages with a tumor antigen; treating the treated macrophages with a selective HDAC6 inhibitor; collecting the STAT-activated macrophages; mixing the STAT-activated macrophages with an adjuvant, diluent, or carrier; administering the STAT-activated macrophages or composition thereof to the subject.

In one aspect, the disease or disorder to be treated/prevented is pulmonary fibrosis.

In another aspect, the disease or disorder to be treated/prevented is liver fibrosis.

In another aspect, the disease or disorder to be treated/prevented is heart fibrosis.

In another aspect, the disease or disorder to be treated/prevented is cancer. STAT-activated macrophages and pharmaceutical compositions comprising STAT-activated macrophages are capable of treating or preventing a cancer, e.g. inhibit the development/progression of the cancer, delay/prevent onset of the cancer, reduce/delay/prevent tumor growth, reduce/delay/prevent metastasis, reduce the severity of the symptoms of the cancer, reduce the number of cancer cells, reduce tumor size/volume, and/or increase survival (e.g. progression free survival).

In one aspect, the cancer is a solid tumor. In another aspect, the cancer is a hematological cancer. In another aspect, the cancer is any one or more of the cancers of Table 3.

TABLE 3

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large | anaplastic thyroid | angioimmunoblastic | angiomyolipoma |
| cell lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial | |
| | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular | follicular thyroid | ganglioneuroma | gastrointestinal |
| lymphoma | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological cancer | hepatoblastoma |
| cancer | | | |
| hepatosplenic T- | Hodgkin's lymphoma | non-Hodgkin's | invasive lobular |
| cell lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone B- | mast cell leukemia | mediastinal germ | medullary |
| cell lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid | myxoma | myxosarcoma | nasopharyngeal |
| liposarcoma | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell | renal medullary | retinoblastoma | rhabdomyoma |
| carcinoma | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

Exemplary hematological cancers include, but are not limited to, the cancers listed in Table 4. In another aspect, the hematological cancer is acute lymphocytic leukemia, chronic lymphocytic leukemia (including B-cell chronic lymphocytic leukemia), or acute myeloid leukemia.

TABLE 4

| | |
|---|---|
| acute lymphocytic leukemia | acute eosinophilic leukemia |
| (ALL) | |
| acute myeloid leukemia (AML) | acute erythroid leukemia |
| chronic lymphocytic leukemia | acute lymphoblastic leukemia |
| (CLL) | |
| small lymphocytic lymphoma | acute megakaryoblastic leukemia |
| (SLL) | |
| multiple myeloma (MM) | acute monocytic leukemia |
| Hodgkins lymphoma (HL) | acute promyelocytic leukemia |
| non-Hodgkin's lymphoma | acute myelogeous leukemia |
| (NHL) | |
| mantle cell lymphoma (MCL) | B-cell prolymphocytic leukemia |
| marginal zone B-cell lymphoma | B-cell lymphoma |
| splenic marginal zone | MALT lymphoma |
| lymphoma | |
| follicular lymphoma (FL) | precursor T-lymphoblastic lymphoma |
| Waldenstrom's | T-cell lymphoma |
| macroglobulinemia (WM) | |
| diffuse large B-cell lymphoma | mast cell leukemia |
| (DLBCL) | |
| marginal zone lymphoma | adult T cell leukemia/lymphoma |
| (MZL) | |
| hairy cell leukemia (HCL) | aggressive NK-cell leukemia |
| Burkitt's lymphoma (BL) | angioimmunoblastic T-cell lymphoma |
| Richter's transformation | |

In one aspect, administration of a STAT-activated macrophage or a composition comprising a STAT-activated macrophage is in a “therapeutically effective” or “prophylactically effective” amount, this being sufficient to show benefit to the subject.

The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Multiple doses of a STAT-activated macrophage or pharmaceutical composition comprising a STAT-activated macrophage may be administered to a subject. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, 6, 7, 8, 9, 10, 11, or 12 months. By way of example, doses may be given once every 7, 14, 21 or 28 days (plus or minus 3, 2, or 1 days).

In another aspect, the present disclosure provides the method further comprising administering to a subject one or more of local radiation therapy, immune checkpoint blockade therapy, photothermal therapy, or chemotherapy.

### HDAC6 Inhibitors

In one aspect, the STAT-activated macrophages are produced from naïve macrophages that have been isolated from a subject and treated ex vivo with a STAT inhibitor and a selective HDAC6 inhibitor.

In another aspect, the selective HDAC6 inhibitor is a compound of Formula I:

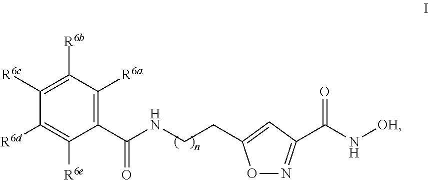

or a pharmaceutically acceptable salt thereof, wherein:
R^{6a}, R^{6b}, R^{6c}, R^{6d}, and R^{6e} are each independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₆ cycloalkyl, optionally substituted phenyl, optionally substituted 5- or 6-membered heteroaryl, and optionally substituted 5- or 6-membered heterocyclo;
R^{a} and R^{b} are independently selected from the group consisting of hydrogen and C₁₋₄ alkyl; or
R^{a} and R^{b} taken together with the nitrogen atom to which they are attached form a 3- to 10-membered heterocyclo;
R^{c} is C₁₋₄ alkyl; and
n is 1, 2, or 3.

In another aspect, the present disclosure provides that the selective HDAC6 inhibitor is a compound of Formula I, or a pharmaceutically acceptable salt thereof, wherein R^{6a}, R^{6b}, R^{6c}, R^{6d}, and R^{6e} are each independently selected from the group consisting of hydrogen, halogen, hydroxy, nitro, cyano, —NR^{a}R^{b}, —C(═O)NR^{a}R^{b}, —C(═O)R^{c}, C₁₋₄ alkyl, C₁₋₄ alkoxy, and C₁₋₄ haloalkyl. In another aspect, R^{6a}, R^{6b}, R^{6c}, R^{6d}, and R^{6e} are each independently selected from the group consisting of hydrogen, halogen, cyano, C₁₋₄ alkyl, and C₁₋₄ alkoxy.

In another aspect, the present disclosure provides that the selective HDAC6 inhibitor is a compound of Formula I, or a pharmaceutically acceptable salt thereof, wherein n is 1. In another aspect, n is 2. In another aspect, n is 3.

In another aspect, the selective HDAC6 inhibitor is a compound of Formula II:

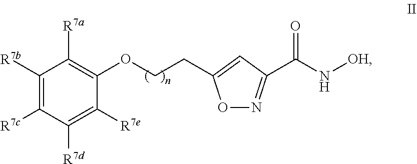

or a pharmaceutically acceptable salt thereof, wherein:
R^{7a}, R^{7b}, R^{7c}, R^{7d}, and R^{7e} are each independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₆ cycloalkyl, optionally substituted phenyl, optionally substituted 5- or 6-membered heteroaryl, and optionally substituted 5- or 6-membered heterocyclo;
R^{a} and R^{b} are independently selected from the group consisting of hydrogen and C₁₋₄ alkyl; or
R^{a} and R^{b} taken together with the nitrogen atom to which they are attached form a 3- to 10-membered heterocyclo;
R^{c} is C₁₋₄ alkyl; and
n is 1, 2, or 3.

In another aspect, the present disclosure provides that the selective HDAC6 inhibitor is a compound of Formula II, or a pharmaceutically acceptable salt thereof, wherein R^{7a}, R^{7b}, R^{7c}, R^{7d}, and R^{7e} are each independently selected from the group consisting of hydrogen, halogen, hydroxy, nitro, cyano, —NR^{a}R^{b}, —C(═O)NR^{a}R^{b}, —C(═O)R^{c}, C₁₋₄ alkyl, C₁₋₄ alkoxy, and C₁₋₄ haloalkyl. In another aspect, R^{7a}, R^{7b}, R^{7c}, R^{7d}, and R^{7e} are each independently selected from the group consisting of hydrogen, halogen, cyano, C₁₋₄ alkyl, and C₁₋₄ alkoxy.

In another aspect, the selective HDAC6 inhibitor is a compound of Formula II, or a pharmaceutically acceptable salt thereof, wherein n is 1. In another aspect, n is 2. In another aspect, n is 3.

In another aspect, the selective HDAC6 inhibitor is a compound of Formula III:

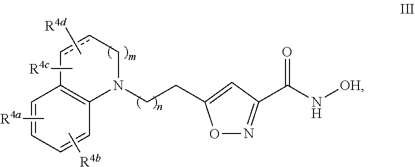

or a pharmaceutically acceptable salt thereof, wherein:
R^{4a} and R^{4b} are independently selected from the group consisting of hydrogen, halogen, cyano, C₁₋₄ alkyl, and C₁₋₄ alkoxy;
R^{4c} and R^{4d} are independently selected from the group consisting of hydrogen and methyl;
m is 0 or 1;
n is 1, 2, or 3; and
[symbol] represents a single or double bond.

In another aspect, the selective HDAC6 inhibitor is a compound of Formula III, or a pharmaceutically acceptable salt thereof, wherein m is 0 and [symbol] can represent a double bond.

In another aspect, the selective HDAC6 inhibitor is a compound of Formula III, or a pharmaceutically acceptable salt thereof, wherein m is 1 and [symbol] is a single bond.

In another aspect, the selective HDAC6 inhibitor is a compound of Formula III, or a pharmaceutically acceptable salt thereof, wherein n is 1. In another aspect, n is 2. In another aspect, n is 3.

In another aspect, the selective HDAC6 inhibitor that is a compound of Formula IV:

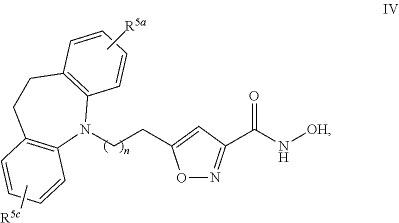

or a pharmaceutically acceptable salt thereof, wherein:
R^{5a} and R^{5c} are independently selected from the group consisting of hydrogen, halogen, cyano, C₁₋₄ alkyl, and C₁₋₄ alkoxy; and
n is 1, 2, or 3.

In another aspect, the selective HDAC6 inhibitor is a compound of Formula IV, or a pharmaceutically acceptable salt thereof, wherein n is 1. In another aspect, n is 2. In another aspect, n is 3.

In another aspect, the selective HDAC6 inhibitor is a compound of Table 2, see below, or a pharmaceutically acceptable salt thereof.

In another aspect, the selective HDAC6 inhibitor is at least 20-fold selective over one or more other HDAC isoforms, e.g., HDAC1, HDAC2, HDAC3, HDAC4, HDAC5, HDAC7, HDAC8, HDAC9, HDAC10, or HDAC11.

In another aspect, the selective HDAC6 inhibitor is at least 100-fold selective over one or more other HDAC isoforms.

In another aspect, the selective HDAC6 inhibitor is at least 600-fold selective over one or more other HDAC isoforms.

### Radiation Therapy

In one aspect, methods provided herein comprise administering STAT-activated macrophages or a composition comprising STAT-activated macrophages to a subject in combination with radiation therapy. The methods provided herein are not limited by the types, amounts, or delivery and administration systems used to deliver the therapeutic dose of radiation to a subject. For example, the subject may receive photon radiotherapy, particle beam radiation therapy, other types of radiotherapies, and combinations thereof. In some aspects, the radiation is delivered to the subject using a linear accelerator. In still other aspects, the radiation is delivered using a gamma knife.

The source of radiation can be external or internal to the subject. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by subjects. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The subject may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a subject, so long as the dose of radiation is tolerated by the subject without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one aspect, the dose of radiation is fractionated for maximal target cell exposure and reduced toxicity.

In one aspect, the total dose of radiation administered to a subject is about 0.01 Gray (Gy) to about 100 Gy. In another aspect, about 10 Gy to about 65 Gy (e.g., about 15 Gy, Gy, 25 Gy, 30 Gy, 35 Gy, 40 Gy, 45 Gy, 50 Gy, 55 Gy, or 60 Gy) are administered over the course of treatment. While in some aspects a complete dose of radiation can be administered over the course of one day, the total dose is ideally fractionated and administered over several days. Desirably, radiotherapy is administered over the course of at least about 3 days, e.g., at least 3, 4, 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one aspect, radiation is not administered every day, thereby allowing the animal to rest and the effects of the therapy to be realized. For example, in one aspect, radiation is administered on 5 consecutive days, and not administered for 2 days, for each week of treatment, thereby allowing 2 days of rest per week. However, in other aspects, radiation is administered 1 day/week, 2 days/week, 3 days/week, 4 days/week, 5 days/week, 6 days/week, or all 7 days/week, depending on the mammal's responsiveness and any potential side effects. Radiation therapy can be initiated at any time in the therapeutic period. In one aspect, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the methods provided herein.

### Immune Checkpoint Blockade Therapy

In one aspect, methods provided herein comprise administering STAT-activated macrophages or a composition comprising STAT-activated macrophages to a subject in combination with immune checkpoint blockade therapy. Immune checkpoint inhibitors are therapies that block immune system inhibitor checkpoints. Immune checkpoints can be stimulatory or inhibitory. Blockade of inhibitory immune checkpoints activates immune system function and is useful for cancer immunotherapy. Pardoll, Nature Reviews. Cancer 12:252-64 (2012). Tumor cells turn off activated T cells when they attach to specific T-cell receptors. Immune checkpoint inhibitors prevent tumor cells from attaching to T cells, which results in T cells remaining activated. In effect, the coordinated action by cellular and soluble components combats pathogens and injuries by cancers. The modulation of immune system pathways may involve changing the expression or the functional activity of at least one component of the pathway to then modulate the response by the immune system. U.S. 2015/0250853. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one aspect, the immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another aspect, the immune checkpoint inhibitor is a programmed cell death protein (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to, nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another aspect, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another aspect, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another aspect, the immune checkpoint inhibitor is a LAG3 inhibitor. LAG3, Lymphocyte Activation Gene 3, is a negative co-stimulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another aspect, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another aspect, the immune checkpoint inhibitor is a CD47 inhibitor. See Unanue, E. R., PNAS 110:10886-87 (2013).

The term “antibody” is meant to include intact monoclonal antibodies, polyclonal antibodies, and multispecific antibodies formed from at least two intact antibodies, so long as they exhibit the desired biological activity. In one aspect, the antibodies are humanized monoclonal antibodies made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053.

Another class of immune checkpoint inhibitors include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to levo-1-methyl typtophan (L-1MT) and 1-methyl-tryptophan (1MT). Qian et al., Cancer Res 69:5498-504 (2009); and Löb et al., Cancer Immunol Immunother 58.153-7 (2009).

In one aspect, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559, or MED14736.

### Chemotherapy

In one aspect, methods provided herein comprise administering a composition comprising STAT-activated macrophages or a composition comprising STAT-activated macrophages to a subject in combination with chemotherapy. In one aspect, the chemotherapy comprises one of the anti-cancer drugs or anti-cancer drug combinations listed in Table 5.

TABLE 5

| | | | |
|---|---|---|---|
| Abemaciclib | Abiraterone | Abraxane (Paclitaxel | ABVD |
| | Acetate | Albumin-stabilized | |
| | | Nanoparticle | |
| | | Formulation) | |
| ABVE | ABVE-PC | AC | Acalabrutinib |
| AC-T | Actemra | Adcetris (Brentuximab | ADE |
| | (Tocilizumab) | Vedotin) | |
| Ado-Trastuzumab | Adriamycin | Afatinib Dimaleate | Afinitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta (Pemetrexed | Aliqopa |
| | | Disodium) | (Copanlisib |
| | | | Hydrochloride) |
| Alkeran for | Alkeran Tablets | Aloxi (Palonosetron | Alunbrig |
| Injection | (Melphalan) | Hydrochloride) | (Brigatinib) |
| (Melphalan | | | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp (Darbepoetin | Aredia |
| | | Alfa) | (Pamidronate |
| | | | Disodium) |
| Arimidex | Aromasin | Arranon (Nelarabine) | Arsenic Trioxide |
| (Anastrozole) | (Exemestane) | | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane I 131) | (Avelumab) | | (Belinostat) |
| Belinostat | Bendamustine | Bendeka (Bendamustine | BEP |
| | Hydrochloride | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif (Bosutinib) | Bosutinib |
| (Blinatumomab) | | | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S- |
| | | | Malate) |
| Cabozantinib-S- | CAF | Calquence | Campath |
| Malate | | (Acalabrutinib) | (Alemtuzumab) |
| Camptosar | Capecitabine | CAPOX | Carac |
| (Irinotecan | | | (Fluorouracil-- |
| Hydrochloride) | | | Topical) |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix (Recombinant | Cetuximab |
| | (Daunorubicin | HPV Bivalent Vaccine) | |
| | Hydrochloride) | | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq (Cabozantinib- | Copanlisib |
| | | S-Malate) | Hydrochloride |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio (Defibrotide | Degarelix |
| | Sodium | Sodium) | |
| Denileukin | Denosumab | DepoCyt (Cytarabine | Dexamethasone |
| Diftitox | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL (Doxorubicin | Durvalumab |
| Hydrochloride | Hydrochloride | Hydrochloride | |
| | Liposome | Liposome) | |
| Duvelisib | Efudex | Eligard (Leuprolide | Elitek |
| | (Fluorouracil-- | Acetate) | (Rasburicase) |
| | Topical) | | |
| Ellence | Elotuzumab | Eloxatin (Oxaliplatin) | Eltrombopag |
| (Epirubicin | | | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen (Epoetin | Erbitux | Eribulin Mesylate | Erivedge |
| Alfa) | (Cetuximab) | | (Vismodegib) |
| Erleada | Erlotinib | Erwinaze (Asparaginase | Ethyol |
| (Apalutamide) | Hydrochloride | Erwinia chrysanthemi) | (Amifostine) |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista (Raloxifene | Evomela (Melphalan | Exemestane |
| | Hydrochloride) | Hydrochloride) | |
| 5-FU (Fluorouracil | 5-FU | Fareston (Toremifene) | Farydak |
| Injection) | (Fluorouracil-- | | (Panobinostat) |
| | Topical) | | |
| Faslodex | FEC | Femara (Letrozole) | Filgrastim |
| (Fulvestrant) | | | |
| Firmagon | Fludarabine | Fluoroplex (Fluorouracil-- | Fluorouracil |
| (Degarelix) | Phosphate | Topical) | Injection |
| Fluorouracil-- | Flutamide | FOLFIRI | FOLFIRI- |
| Topical | | | BEVACIZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva (Obinutuzumab) | Gefitinib |
| (Recombinant | (Recombinant | | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif (Afatinib | Gleevec (Imatinib | Gliadel Wafer |
| (Gemcitabine | Dimaleate) | Mesylate) | (Carmustine |
| Hydrochloride) | | | Implant) |
| Glucarpidase | Goserelin Acetate | Granisetron | Granisetron |
| | | | Hydrochloride |
| Granix | Halaven (Eribulin | Hemangeol (Propranolol | Herceptin |
| (Filgrastim) | Mesylate) | Hydrochloride) | (Trastuzumab) |
| HPV Bivalent | HPV Nonavalent | HPV Quadrivalent | Hycamtin |
| Vaccine, | Vaccine, | Vaccine, Recombinant | (Topotecan |
| Recombinant | Recombinant | | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa (Enasidenib | Ifex (Ifosfamide) |
| Hydrochloride | | Mesylate) | |
| Ifosfamide | IL-2 | Imatinib Mesylate | Imbruvica |
| | (Aldesleukin) | | (Ibrutinib) |
| Imfinzi | Imiquimod | Imlygic (Talimogene | Inlyta (Axitinib) |
| (Durvalumab) | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa- |
| | | | 2b) |
| Iobenguane I 131 | Ipilimumab | Iressa (Gefitinib) | Irinotecan |
| | | | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi (Ruxolitinib | JEB |
| | (Ixabepilone) | Phosphate) | |
| Jevtana | Kadcyla (Ado- | Kepivance (Palifermin) | Keytruda |
| (Cabazitaxel) | Trastuzumab | | (Pembrolizumab) |
| | Emtansine) | | |
| Kisqali | Kymriah | Kyprolis (Carfilzomib) | Lanreotide |
| (Ribociclib) | (Tisagenlecleucel) | | Acetate |
| Lapatinib | Larotrectinib | Lartruvo (Olaratumab) | Lenalidomide |
| Ditosylate | Sulfate | | |
| Lenvatinib | Lenvima | Letrozole | Leucovorin |
| Mesylate | (Lenvatinib | | Calcium |
| | Mesylate) | | |
| Leukeran | Leuprolide | Levulan | Libtayo |
| (Chlorambucil) | Acetate | Kerastik (Aminolevulinic | (Cemiplimab- |
| | | Acid) | rwlc) |
| LipoDox | Lomustine | Lonsurf (Trifluridine and | Lorbrena |
| (Doxorubicin | | Tipiracil Hydrochloride) | (Lorlatinib) |
| Hydrochloride | | | |
| Liposome) | | | |
| Lorlatinib | Lumoxiti | Lupron (Leuprolide | Lupron Depot |
| | (Moxetumomab | Acetate) | (Leuprolide |
| | Pasudotox-tdfk) | | Acetate) |
| Lutathera | Lutetium (Lu 177- | Lynparza (Olaparib) | Marqibo |
| (Lutetium Lu 177- | Dotatate) | | (Vincristine |
| Dotatate) | | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methylnaltrexone |
| | | | Bromide |
| Midostaurin | Mitomycin C | Mitoxantrone | Mogamulizumab- |
| | | Hydrochloride | kpkc |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib |
| | | | Maleate) |
| Netupitant and | Neulasta | Neupogen (Filgrastim) | Nexavar |
| Palonosetron | (Pegfilgrastim) | | (Sorafenib |
| Hydrochloride | | | Tosylate) |
| Nilandron | Nilotinib | Nilutamide | Ninlaro |
| (Nilutamide) | | | (Ixazomib |
| | | | Citrate) |
| Niraparib Tosylate | Nivolumab | Nplate (Romiplostim) | Obinutuzumab |
| Monohydrate | | | |
| Odomzo | OEPA | Ofatumumab | OFF |
| (Sonidegib) | | | |
| Olaparib | Olaratumab | Omacetaxine | Oncaspar |
| | | Mepesuccinate | (Pegaspargase) |
| Ondansetron | Onivyde | Ontak (Denileukin | Opdivo |
| Hydrochloride | (Irinotecan | Diftitox) | (Nivolumab) |
| | Hydrochloride | | |
| | Liposome) | | |
| OPPA | Osimertinib | Oxaliplatin | Paclitaxel |
| Paclitaxel | PAD | Palbociclib | Palifermin |
| Albumin-stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta (Pertuzumab) | Pertuzumab |
| | Disodium | | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab- | | |
| | kpkc) | | |
| Procarbazine | Procrit (Epoetin | Proleukin (Aldesleukin) | Prolia |
| Hydrochloride | Alfa) | | (Denosumab) |
| Promacta | Propranolol | Provenge (Sipuleucel-T) | Purinethol |
| (Eltrombopag | Hydrochloride | | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant | Recombinant | Recombinant Interferon | Regorafenib |
| Human | Human | Alfa-2b | |
| Papillomavirus | Papillomavirus | | |
| (HPV) Nonavalent | (HPV) | | |
| Vaccine | Quadrivalent | | |
| | Vaccine | | |
| Relistor | R-EPOCH | Retacrit (Epoetin Alfa) | Revlimid |
| (Methylnaltrexone | | | (Lenalidomide) |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase Human | Hydrochloride |
| Hyaluronidase | | | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt (Midostaurin) | Sancuso |
| Camsylate | Phosphate | | (Granisetron) |
| Sclerosol | Siltuximab | Sipuleucel-T | Somatuline Depot |
| Intrapleural | | | (Lanreotide |
| Aerosol (Talc) | | | Acetate) |
| Sonidegib | Sorafenib | Sprycel (Dasatinib) | STANFORD V |
| | Tosylate | | |
| Sterile Talc | Steritalc (Talc) | Stivarga (Regorafenib) | Sunitinib Malate |
| Powder (Talc) | | | |
| Sustol | Sutent (Sunitinib | Sylatron (Peginterferon | Sylvant |
| (Granisetron) | Malate) | Alfa-2b) | (Siltuximab) |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva (Erlotinib | Targretin (Bexarotene) | Tasigna |
| (Cytarabine) | Hydrochloride) | | (Nilotinib) |
| Tavalisse | Taxol (Paclitaxel) | Taxotere (Docetaxel) | Tecentriq |
| (Fostamatinib | | | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak (Fluorouracil-- | Topotecan |
| | | Topical) | Hydrochloride |
| Toremifene | Torisel | Totect (Dexrazoxane | TPF |
| | (Temsirolimus) | Hydrochloride) | |
| Trabectedin | Trametinib | Trastuzumab | Treanda |
| | | | (Bendamustine |
| | | | Hydrochloride) |
| Trexall | Trifluridine and | Trisenox (Arsenic | Tykerb (Lapatinib |
| (Methotrexate) | Tipiracil | Trioxide) | Ditosylate) |
| | Hydrochloride | | |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VeIP | Velcade (Bortezomib) | Vemurafenib |
| (Panitumumab) | | | |
| Venclexta | Venetoclax | Verzenio (Abemaciclib) | Vidaza |
| (Venetoclax) | | | (Azacitidine) |
| Vinblastine Sulfate | Vincristine | Vincristine Sulfate | Vinorelbine |
| | Sulfate | Liposome | Tartrate |
| VIP | Vismodegib | Vistogard (Uridine | Vitrakvi |
| | | Triacetate) | (Larotrectinib |
| | | | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda (Capecitabine) | XELIRI |
| (Daunorubicin | (Crizotinib) | | |
| Hydrochloride and | | | |
| Cytarabine | | | |
| Liposome) | | | |
| XELOX | Xgeva | Xofigo (Radium 223 | Xtandi |
| | (Denosumab) | Dichloride) | (Enzalutamide) |
| Yervoy | Yescarta | Yondelis (Trabectedin) | Zaltrap (Ziv- |
| (Ipilimumab) | (Axicabtagene | | Aflibercept) |
| | Ciloleucel) | | |
| Zarxio (Filgrastim) | Zejula (Niraparib | Zelboraf (Vemurafenib) | Zevalin |
| | Tosylate | | (Ibritumomab |
| | Monohydrate) | | Tiuxetan) |
| Zinecard | Ziv-Aflibercept | Zofran (Ondansetron | Zoladex |
| (Dexrazoxane | | Hydrochloride) | (Goserelin |
| Hydrochloride) | | | Acetate) |
| Zoledronic Acid | Zolinza | Zometa (Zoledronic | Zydelig |
| | (Vorinostat) | Acid) | (Idelalisib) |
| Zykadia | Zytiga | | |
| (Ceritinib) | (Abiraterone | | |
| | Acetate) | | |

### Photothermal Therapy

In one aspect, methods provided herein comprise administering STAT-activated macrophages or a composition comprising STAT-activated macrophages to a subject in combination with photothermal therapy. Photothermal therapy refers to efforts to use electromagnetic radiation (most often in infrared wavelengths) for the treatment of various medical conditions, including cancer. This approach is an extension of photodynamic therapy, in which a photosensitizer is excited with specific band light. This activation brings the sensitizer to an excited state where it then releases vibrational energy (heat), which is what kills the targeted cells. Unlike photodynamic therapy, photothermal therapy does not require oxygen to interact with the target cells or tissues. Current studies also show that photothermal therapy is able to use longer wavelength light, which is less energetic and therefore less harmful to other cells and tissues.

Most materials of interest currently being investigated for photothermal therapy are on the nanoscale. One of the key reasons behind this is the enhanced permeability and retention effect observed with particles in a certain size range (typically 20-300 nm). Maeda et. al., Journal of Controlled Release, 65 (1-2), 271-284 (2000). Molecules in this range have been observed to preferentially accumulate in tumor tissue. When a tumor forms, it requires new blood vessels in order to fuel its growth; these new blood vessels in/near tumors have different properties as compared to regular blood vessels, such as poor lymphatic drainage and a disorganized, leaky vasculature. These factors lead to a significantly higher concentration of certain particles in a tumor as compared to the rest of the body. Coupling this phenomenon with active targeting modalities (e.g., antibodies) has recently been investigated by researchers.

### IV. Definitions

The term “STAT-activated macrophage” refers to a naïve macrophage that has been treated ex vivo with a STAT inhibitor. In another aspect, the STAT-activated macrophage is first treated ex vivo with a STAT inhibitor and then treated ex vivo with a macrophage polarizing agent and/or tumor antigen and/or a selective HDAC6 inhibitor. In another aspect, the STAT-activated macrophage is first treated ex vivo with a macrophage polarizing agent and/or tumor antigen and/or a selective HDAC6 inhibitor and then treated ex vivo with a STAT inhibitor.

The STAT protein family is composed of seven members: STAT1, STAT2, STAT3, STAT4, STAT5A, STAT5B, and STATE.

In one aspect, the STAT-activated macrophages are produced from naïve macrophages that have been isolated from a subject and treated ex vivo with a STAT1 inhibitor.

In another aspect, the STAT-activated macrophages are produced from naïve macrophages that have been isolated from a subject and treated ex vivo with a STAT2 inhibitor.

In another aspect, the STAT-activated macrophages are produced from naïve macrophages that have been isolated from a subject and treated ex vivo with a STAT3 inhibitor.

In another aspect, the STAT-activated macrophages are produced from naïve macrophages that have been isolated from a subject and treated ex vivo with a STAT4 inhibitor.

In another aspect, the STAT-activated macrophages are produced from naïve macrophages that have been isolated from a subject and treated ex vivo with a STAT5A inhibitor.

In another aspect, the STAT-activated macrophages are produced from naïve macrophages that have been isolated from a subject and treated ex vivo with a STAT5B inhibitor.

In another aspect, the STAT-activated macrophages are produced from naïve macrophages that have been isolated from a subject and treated ex vivo with a STATE inhibitor.

The terms “selective HDAC6 inhibitor,” “HDAC6 selective inhibitor,” and the like as used herein refer to a compound that preferentially inhibits histone deacetylase 6 over one or more other histone deacetylase isoforms, e.g., HDAC1, HDAC2, HDAC3, HDAC4, HDAC5, HDAC7, HDAC8, HDAC9, HDAC10, and/or HDAC11, in a cell-based in vitro assay. For example, a compound having a HDAC6 IC₅₀=5 nM and a HDAC1 IC₅₀ of 500 nM is a selective HDAC6 inhibitor that is 100-fold more selective over HDAC1; a compound having a HDAC6 IC₅₀=5 nM, a HDAC1 IC₅₀=500 nM, and a HDAC3 IC₅₀=50 nM is a selective HDAC6 inhibitor that is 100-fold more selective over HDAC1 and 10-fold more selective over HDAC3; and so on. In one aspect, the selective HDAC6 inhibitor preferentially inhibits HDAC6 over HDAC1. In another aspect, the selective HDAC6 inhibitor preferentially inhibits HDAC6 over HDAC1 and one or more other HDAC isoforms.

In one aspect, the selective HDAC6 inhibitor is at least about 5-fold more selective over one or more other HDAC isoforms. In another aspect, the selective HDAC6 inhibitor is at least about 10-fold more selective over one or more other HDAC isoforms. In another aspect, the selective HDAC6 inhibitor is at least about 15-fold more selective over one or more other HDAC isoforms. In another aspect, the selective HDAC6 inhibitor is at least about 20-fold more selective over one or more other HDAC isoforms. In another aspect, the selective HDAC6 inhibitor is at least about 30-fold more selective over one or more other HDAC isoforms. In another aspect, the selective HDAC6 inhibitor is at least about more selective over one or more other HDAC isoforms. In another aspect, the selective HDAC6 inhibitor is at least about 50-fold more selective over one or more other HDAC isoforms. In another aspect, the selective HDAC6 inhibitor is at least about 100-fold more selective over one or more other HDAC isoforms. In another aspect, the selective HDAC6 inhibitor is at least about 150-fold more selective over one or more other HDAC isoforms. In another aspect, the selective HDAC6 inhibitor is at least about 200-fold more selective over one or more other HDAC isoforms. In another aspect, the selective HDAC6 inhibitor is at least about 250-fold more selective over one or more other HDAC isoforms. In another aspect, the selective HDAC6 inhibitor is at least about 500-fold more selective over one or more other HDAC isoforms. In another aspect, the selective HDAC6 inhibitor is at least about 750-fold more selective over one or more other HDAC isoforms. In another aspect, the selective HDAC6 inhibitor is at least about 1000-fold more selective over one or more other HDAC isoforms. In another aspect, the selective HDAC6 inhibitor is at least about 2000-fold more selective over one or more other HDAC isoforms. In another aspect, the selective HDAC6 inhibitor is at least about 3000-fold more selective over one or more other HDAC isoforms. HDAC6 selectivity over the other HDAC isoforms in cell-based assays can be determined using methods known in the art.

In another aspect, the selective HDAC6 inhibitor is at about 10-fold to about 3000-fold more selective over one or more other HDAC isoforms. In another aspect, the selective HDAC6 inhibitor is at about 20-fold to about 3000-fold more selective over one or more other HDAC isoforms. In another aspect, the selective HDAC6 inhibitor is at about to about 3000-fold more selective over one or more other HDAC isoforms. In another aspect, the selective HDAC6 inhibitor is at about 100-fold to about 3000-fold more selective over one or more other HDAC isoforms. In another aspect, the selective HDAC6 inhibitor is at about 500-fold to about 3000-fold more selective over one or more other HDAC isoforms.

In one aspect, HDAC6 selectivity is determined using an isolated human, recombinant full-length HDAC from a baculovirus expression system in Sf9 cells. An acetylated fluorogenic peptide is used as the substrate depending on the HDAC isoform that is being tested, e.g., one derived from residues 379-382 of p53. See http://www.reactionbiology.com/webapps/site/HDACAssay.aspx?page=HDACs&id=-%203. The reaction buffer is made up of 50 mM Tris-HCl pH 8.0, 127 mM NaCl, 2.7 mM KCl, 1 mM MgCl₂, 1 mg/mL BSA, and a final concentration of 1% DMSO. The test compound is delivered in DMSO to the enzyme mixture with a pre-incubation of 5-10 min followed by substrate addition and incubation for 2 h at 30° C. Trichostatin A and developer are added to quench the reaction and generate fluorescence, respectively. A dose-response curve is generated and the IC₅₀ value is determined from the resulting plot. See Bergman et al., J Med Chem. 55:9891-9899 (2012). The selective HDAC6 inhibitor is meant to include the parent compound and any pharmaceutically acceptable salts or solvates thereof.

In one aspect, the selective HDAC6 inhibitor is a compound disclosed in Shen and Kozikowski, Expert Opinion on Therapeutic Patents 30:121-136 (2020).

In another aspect, the selective HDAC6 inhibitor is a compound disclosed in Bergman et al., J Med Chem. 55:9891-9899 (2012).

In another aspect, the selective HDAC6 inhibitor is a compound disclosed in WO 2014072714.

In another aspect, the selective HDAC6 inhibitor is a compound disclosed in WO 2016067040.

In another aspect, the selective HDAC6 inhibitor is a compound disclosed in WO 2016190630.

In another aspect, the selective HDAC6 inhibitor is a compound disclosed in WO 2019139921.

In another aspect, the selective HDAC6 inhibitor is a compound disclosed in US 20150239869.

In another aspect, the selective HDAC6 inhibitor is a compound disclosed in WO 2015054474.

In another aspect, the selective HDAC6 inhibitor is a compound disclosed in WO 2017075192.

In another aspect, the selective HDAC6 inhibitor is a compound disclosed in WO 2018089651.

In another aspect, the selective HDAC6 inhibitor is a compound disclosed in WO 2014181137.

In another aspect, the selective HDAC6 inhibitor is a compound disclosed in WO 2016067038.

In another aspect, the selective HDAC6 inhibitor is a compound disclosed in WO 2017208032.

In another aspect, the selective HDAC6 inhibitor is a compound disclosed in WO 2016168598.

In another aspect, the selective HDAC6 inhibitor is a compound disclosed in WO 2016168660.

In another aspect, the selective HDAC6 inhibitor is a compound disclosed in WO 2017218950.

In another aspect, the selective HDAC6 inhibitor is a compound disclosed in US 20160221973.

In another aspect, the selective HDAC6 inhibitor is a compound disclosed in US 20160222022.

In another aspect, the selective HDAC6 inhibitor is a compound disclosed in US 20160221997.

In another aspect, the selective HDAC6 inhibitor is a compound disclosed in WO 2014178606.

In another aspect, the selective HDAC6 inhibitor is a compound disclosed in WO 2015087151.

In another aspect, the selective HDAC6 inhibitor is a compound disclosed in WO 2015102426.

In another aspect, the selective HDAC6 inhibitor is a compound disclosed in WO 2015137750.

In another aspect, the selective HDAC6 inhibitor is a compound disclosed in WO 2018189340.

In another aspect, the selective HDAC6 inhibitor is a compound disclosed in WO 2018130155.

In another aspect, the selective HDAC6 inhibitor is a compound disclosed in WO 2017222950.

In another aspect, the selective HDAC6 inhibitor is a compound disclosed in WO 2017222951.

In another aspect, the selective HDAC6 inhibitor is a compound disclosed in WO 2017222952.

In another aspect, the selective HDAC6 inhibitor is a compound disclosed in WO 2016031815.

In another aspect, the selective HDAC6 inhibitor is a compound disclosed in WO 2017014170.

In another aspect, the selective HDAC6 inhibitor is a compound disclosed in WO 2017014321.

In another aspect, the selective HDAC6 inhibitor is a compound disclosed in WO 2017033946.

In another aspect, the selective HDAC6 inhibitor is a compound disclosed in WO 2019027054.

In another aspect, the selective HDAC6 inhibitor is a compound disclosed in WO 2019166824.

In another aspect, the selective HDAC6 inhibitor is a compound disclosed in WO 2019110663.

In another aspect, the selective HDAC6 inhibitor is a compound disclosed in WO 2017018803.

In another aspect, the selective HDAC6 inhibitor is a compound disclosed in WO 2017018805.

In another aspect, the selective HDAC6 inhibitor is a compound disclosed in WO 2017023133.

In another aspect, the selective HDAC6 inhibitor is a compound disclosed in WO 2017065473.

In another aspect, the selective HDAC6 inhibitor is a compound disclosed in WO 2018183701.

In another aspect, the selective HDAC6 inhibitor is a compound having Formula V:

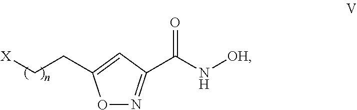

wherein:
X is selected from the group consisting of:

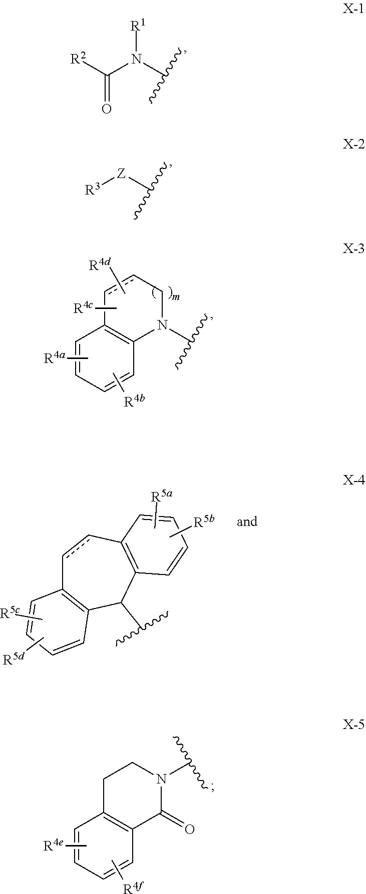

R¹ is selected from the group consisting of hydrogen and C₁₋₄ alkyl;
R² is selected from the group consisting of optionally substituted C₆-C₁₄ aryl and aralkyl;
R³ is selected from the group consisting of optionally substituted C₆-C₁₄ aryl, optionally substituted 5- to 14-membered heteroaryl, and —C(═O)NR^{d}R^{e};
R^{4a}, R^{4b}, R^{4e} and R^{4f} are independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, and haloalkoxy;
R^{4c} and R^{4d} are independently selected from the group consisting of hydrogen and C₁₋₄ alkyl; or
R^{4c} and R^{4d} taken together form a —C(═O)— with the carbon atom to which they are attached;
R^{5a}, R^{5b}, R^{5c}, and R^{5d} are independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, and haloalkoxy;
Z is selected from the group consisting of —O—, —N(R⁸)—, and —C(═O)—; or
Z is absent;
R⁸ is selected from the group consisting of hydrogen, C₁₋₄ alkyl, optionally substituted C₃₋₆ cycloalkyl, optionally substituted C₆-C₁₄ aryl, aralkyl, optionally substituted 5- to 14-membered heteroaryl, and heteroaralkyl;
m is 0, 1, or 2;
n is 1, 2, 3, 4, 5, or 6;
[symbol] represents a single or double bond;
R^{a}, R^{b}, R^{d}, and R^{e} are independently selected from the group consisting of hydrogen, C₁₋₆ alkyl, optionally substituted C₃₋₆ cycloalkyl, optionally substituted C₆-C₁₄ aryl, optionally substituted 5- to 14-membered heteroaryl; or
R^{a} and R^{b} taken together with the nitrogen atom to which they are attached form an optionally substituted 3- to 12-membered heterocyclo;
R^{d} and R^{e} taken together with the nitrogen atom to which they are attached form an optionally substituted 3- to 12-membered heterocyclo; and
R^{c} is C₁₋₄ alkyl.

In another aspect, the selective HDAC6 inhibitor is a compound having Formula V, and the pharmaceutically acceptable salts, and solvates thereof, with the proviso that when Z is absent, R³ is a bicyclic or tricyclic C₁₀₋₁₄ aryl, a bicyclic or tricyclic 9- to 14-membered heteroaryl, or —C(═O)NR d R^{e}.

In another aspect, the selective HDAC6 inhibitor is a compound having Formula V, and the pharmaceutically acceptable salts, and solvates thereof, wherein X is X-1, X-2, X-3, or X-4;
Z is —O—;
R¹ is selected from the group consisting of hydrogen and C₁₋₄ alkyl;
R² is optionally substituted C₆-C₁₄ aryl;
R³ is selected from the group consisting of optionally substituted C₆-C₁₄ aryl and optionally substituted 5- to 14-membered heteroaryl;
R^{4a} and R^{4b} are independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, and haloalkoxy;
R^{4c} and R^{4d} are independently selected from the group consisting of hydrogen and C₁₋₄ alkyl;
R^{5a}, R^{5b}, R^{5c}, and R^{5d} are independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, and haloalkoxy;
R^{a} and R^{b} are independently selected from the group consisting of hydrogen and C₁₋₆ alkyl; or
R^{a} and R^{b} taken together with the nitrogen atom to which they are attached form a 3- to 7-membered heterocyclo; and
R^{c} is C₁₋₄ alkyl.

In another aspect, the selective HDAC6 inhibitor is a compound having Formula V, wherein X is X-1. In another aspect, R¹ is hydrogen. In another aspect, R² is optionally substituted phenyl. In another aspect, R² is optionally substituted 1-naphthyl. In another aspect, R² is optionally substituted 2-naphthyl. In another aspect, R² is aralkyl.

In another aspect, the selective HDAC6 inhibitor is a compound having Formula V, wherein X is X-2. In another aspect, Z is —O—. In another aspect, Z is —N(R⁸)— In another aspect, Z is —C(═O)—. In another aspect, R³ is optionally substituted C₆-C₁₄ aryl. In another aspect, R³ is optionally substituted 5- to 14-membered heteroaryl. In another aspect, R³ is —C(═O)NR^{d}R^{e}. In another aspect, Z is absent and R³ is a bicyclic or tricyclic C₁₀₋₁₄ aryl, a bicyclic or tricyclic 9- to 14-membered heteroaryl, or —C(═O)NR^{d}R^{e}.

In another aspect, the selective HDAC6 inhibitor is a compound having Formula V, wherein X is X-3.

In another aspect, the selective HDAC6 inhibitor is a compound having Formula V, wherein X is X-4.

In another aspect, the selective HDAC6 inhibitor is a compound having Formula V, wherein X is X-5.

In another aspect, the selective HDAC6 inhibitor is a compound having Formula I:

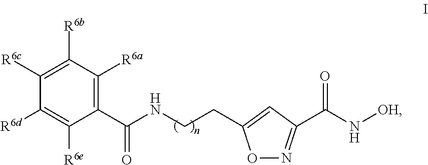

or a pharmaceutically acceptable salt thereof, wherein:
R^{6a}, R^{6b}, R^{6c}, R^{6d}, and R^{6e} are each independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₆ cycloalkyl, optionally substituted phenyl, optionally substituted 5- or 6-membered heteroaryl, and optionally substituted 5- or 6-membered heterocyclo;
R^{a} and R^{b} are independently selected from the group consisting of hydrogen and C₁₋₄ alkyl; or
R^{a} and R^{b} taken together with the nitrogen atom to which they are attached form a 3- to 7-membered heterocyclo;
R^{c} is C₁₋₄ alkyl; and
n is 1, 2, or 3.

In another aspect, the selective HDAC6 inhibitor is a compound having Formula I, wherein R^{6a}, R^{6b}, R^{6c}, R^{6d}, and R^{6e} are each independently selected from the group consisting of hydrogen, halogen, hydroxy, nitro, cyano, —NR^{a}R^{b}, —C(═O)NR^{a}R^{b}, —C(═O)R^{c}, C₁₋₄ alkyl, C₁₋₄ alkoxy, and C₁₋₄ haloalkyl. In another aspect, R^{6a}, R^{6b}, R^{6c}, R^{6d}, and R^{6e} are each independently selected from the group consisting of hydrogen, halogen, cyano, C₁₋₄ alkyl, and C₁₋₄ alkoxy.

In another aspect, the selective HDAC6 inhibitor is a compound having Formula I, wherein n is 1. In another aspect, n is 2. In another aspect, n is 3.

In another aspect, the selective HDAC6 inhibitor is a compound having Formula II:

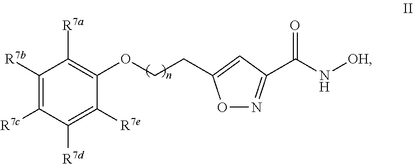

or a pharmaceutically acceptable salt thereof, wherein:
R^{7a}, R^{7b}, R^{7c}, R^{7d}, and R^{7e} are each independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₆ cycloalkyl, optionally substituted phenyl, optionally substituted 5- or 6-membered heteroaryl, and optionally substituted 5- or 6-membered heterocyclo;
R^{a} and R^{b} are independently selected from the group consisting of hydrogen and C₁₋₄ alkyl; or
R^{a} and R^{b} taken together with the nitrogen atom to which they are attached form a 3- to 7-membered heterocyclo;
R^{c} is C₁₋₄ alkyl; and
n is 1, 2, or 3.

In another aspect, the selective HDAC6 inhibitor is a compound having Formula II, wherein R^{7a}, R^{7b}, R^{7c}, R^{7d}, and R^{7e} are each independently selected from the group consisting of hydrogen, halogen, hydroxy, nitro, cyano, —NR^{a}R^{b}, —C(═O)NR^{a}R^{b}, —C(═O)R^{c}, C₁₋₄ alkyl, C₁₋₄ alkoxy, and C₁₋₄ haloalkyl. In another aspect, R^{7a}, R^{7b}, R^{7c}, R^{7d}, and R^{7e} are each independently selected from the group consisting of hydrogen, halogen, cyano, C₁₋₄ alkyl, and C₁₋₄ alkoxy.

In another aspect, the selective HDAC6 inhibitor is a compound having Formula II, wherein n is 1. In another aspect, n is 2. In another aspect, n is 3.

In another aspect, the selective HDAC6 inhibitor is a compound having Formula III:

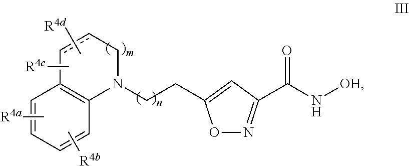

or a pharmaceutically acceptable salt thereof, wherein:
R^{4a} and R^{4b} are independently selected from the group consisting of hydrogen, halogen, cyano, C₁₋₄ alkyl, and C₁₋₄ alkoxy;
R^{4c} and R^{4d} are independently selected from the group consisting of hydrogen and methyl;
m is 0 or 1;
n is 1, 2, or 3; and
[symbol] represents a single or double bond.

In another aspect, the selective HDAC6 inhibitor is a compound having Formula III, wherein m is 0 and [symbol] represents a double bond.

In another aspect, the selective HDAC6 inhibitor is a compound having Formula III, wherein m is 1 and [symbol] represents a single bond.

In another aspect, the selective HDAC6 inhibitor is a compound having Formula III, wherein n is 1. In another aspect, n is 2. In another aspect, n is 3.

In another aspect, the selective HDAC6 inhibitor is a compound having Formula IV:

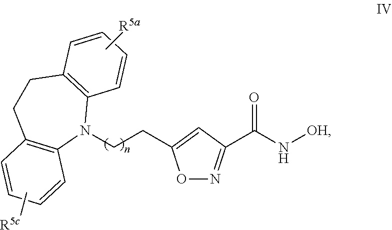

or a pharmaceutically acceptable salt thereof, wherein:
R^{5a} and R^{5c} are independently selected from the group consisting of hydrogen, halogen, cyano, C₁₋₄ alkyl, and C₁₋₄ alkoxy; and
n is 1, 2, or 3.

In another aspect, the selective HDAC6 inhibitor is a compound having Formula IV, wherein n is 1. In another aspect, n is 2. In another aspect, n is 3.

In another aspect, the selective HDAC6 inhibitor is a compound of Table 2, or a pharmaceutically acceptable salt thereof.

TABLE 2

| | Name of | |
|---|---|---|
| Compound | Compound | Structure |
| 1 | Nexturastat A (NextA) | 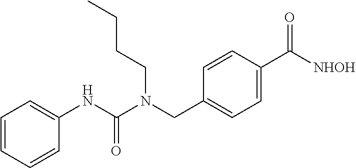 |
| 2 | ACY-1215 | 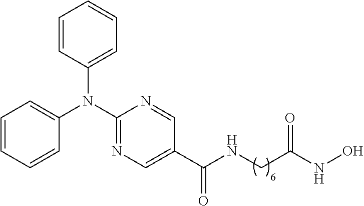 |
| 3 | ACY-241 | 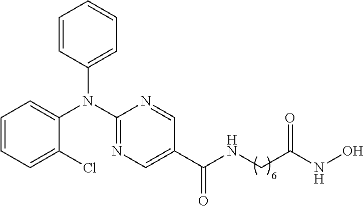 |
| 4 | ACY-738 | 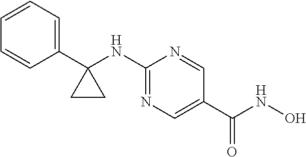 [SMILES: O=C(NO)c1cnc(NC2(c3ccccc3)CC2)nc1] |
| 5 | ACY-775 | 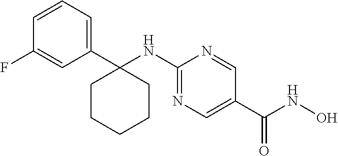 [SMILES: O=C(NO)c1cnc(NC2(c3cccc(F)c3)CCCCC2)nc1] |
| 6 | Tubastatin A | 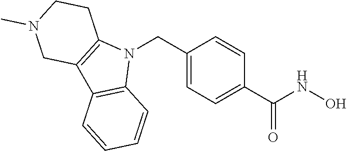 [SMILES: CN1CCc2c(c3ccccc3n2Cc2ccc(C(=O)NO)cc2)C1] |
| 7 | Benzimidazole 23d | 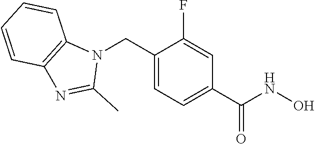 [SMILES: Cc1nc2ccccc2n1Cc1ccc(C(=O)NO)cc1F] |
| 8 | SW-100 | 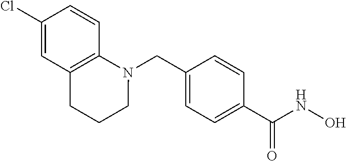 [SMILES: O=C(NO)c1ccc(CN2CCCc3cc(Cl)ccc32)cc1] |
| 9 | W-2 | 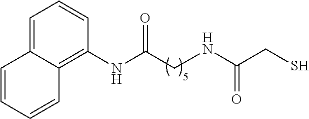 |
| 10 | 5-Aroylindole 6 | 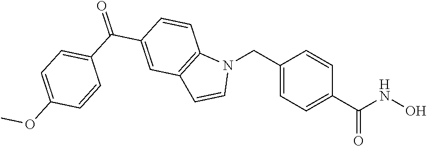 [SMILES: COc1ccc(C(=O)c2ccc3c(ccn3Cc3ccc(C(=O)NO)cc3)c2)cc1] |
| 11 | MPT0G211 | 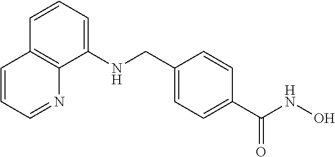 [SMILES: O=C(NO)c1ccc(CNc2cccc3cccnc23)cc1] |
| 12 | | 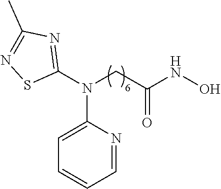 |
| 13 | | 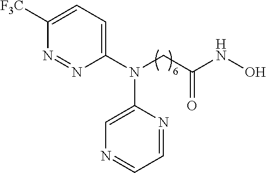 |
| 14 | | 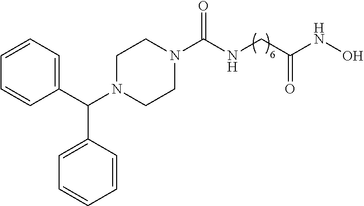 |
| 15 | SP-2-225 | 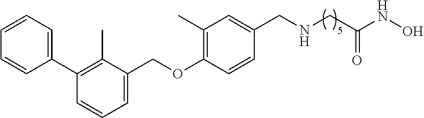 |
| 16 | ACY-1083 | 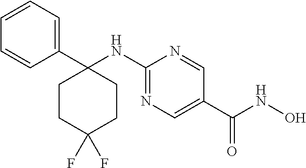 [SMILES: O=C(NO)c1cnc(NC2(c3ccccc3)CCC(F)(F)CC2)nc1] |
| 17 | | 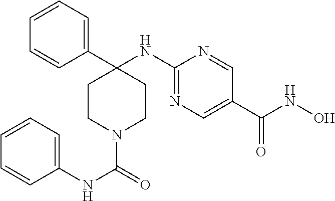 [SMILES: O=C(NO)c1cnc(NC2(c3ccccc3)CCN(C(=O)Nc3ccccc3)CC2)nc1] |
| 18 | | 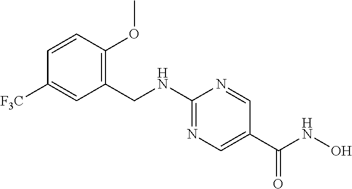 [SMILES: COc1ccc(C(F)(F)F)cc1CNc1ncc(C(=O)NO)cn1] |
| 19 | | 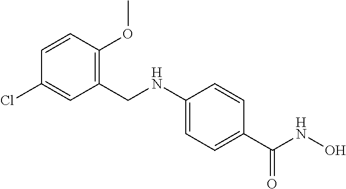 [SMILES: COc1ccc(Cl)cc1CNc1ccc(C(=O)NO)cc1] |
| 20 | | 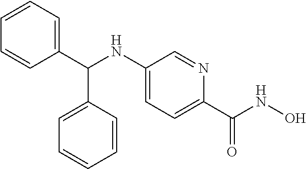 [SMILES: O=C(NO)c1ccc(NC(c2ccccc2)c2ccccc2)cn1] |
| 21 | | 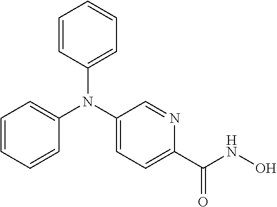 [SMILES: O=C(NO)c1ccc(N(c2ccccc2)c2ccccc2)cn1] |
| 22 | | 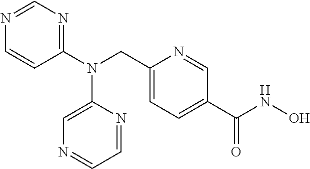 [SMILES: O=C(NO)c1ccc(CN(c2ccncn2)c2cnccn2)nc1] |
| 23 | | 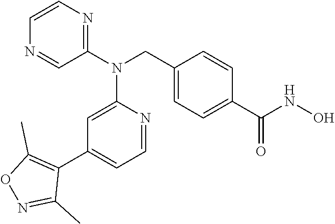 [SMILES: Cc1noc(C)c1-c1ccnc(N(Cc2ccc(C(=O)NO)cc2)c2cnccn2)c1] |
| 24 | | 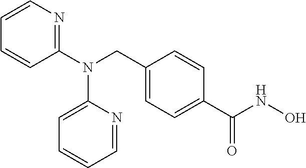 [SMILES: O=C(NO)c1ccc(CN(c2ccccn2)c2ccccn2)cc1] |
| 25 | | 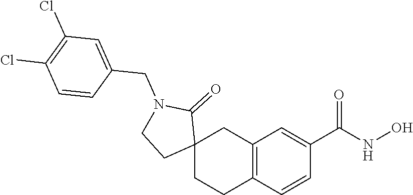 [SMILES: O=C(NO)c1ccc2c(c1)CC1(CC2)CCN(Cc2ccc(Cl)c(Cl)c2)C1=O] |
| 26 | | 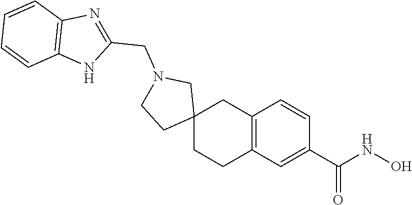 [SMILES: O=C(NO)c1ccc2c(c1)CCC1(CCN(Cc3nc4ccccc4[nH]3)C1)C2] |
| 27 | | 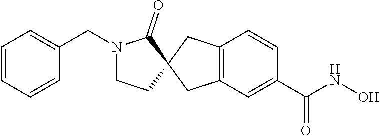 [SMILES: O=C(NO)c1ccc2c(c1)C[C@]1(CCN(Cc3ccccc3)C1=O)C2] |
| 28 | | 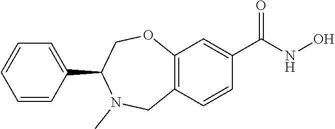 [SMILES: CN1Cc2ccc(C(=O)NO)cc2OC[C@@H]1c1ccccc1] |
| 29 | | 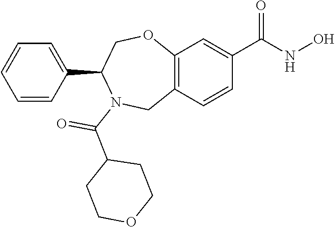 [SMILES: O=C(NO)c1ccc2c(c1)OC[C@H](c1ccccc1)N(C(=O)C1CCOCC1)C2] |
| 30 | | 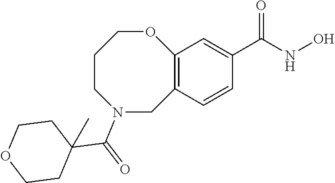 [SMILES: CC1(C(=O)N2CCCOc3cc(C(=O)NO)ccc3C2)CCOCC1] |
| 31 | | 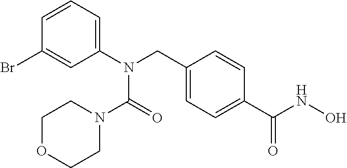 [SMILES: O=C(NO)c1ccc(CN(C(=O)N2CCOCC2)c2cccc(Br)c2)cc1] |
| 32 | | 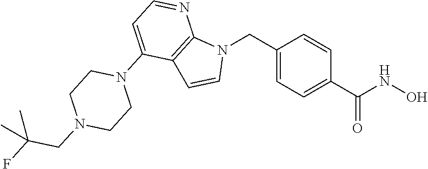 [SMILES: CC(C)(F)CN1CCN(c2ccnc3c2ccn3Cc2ccc(C(=O)NO)cc2)CC1] |
| 33 | | 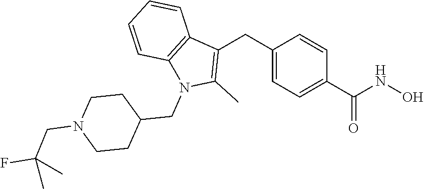 [SMILES: Cc1c(Cc2ccc(C(=O)NO)cc2)c2ccccc2n1CC1CCN(CC(C)(C)F)CC1] |
| 34 | | 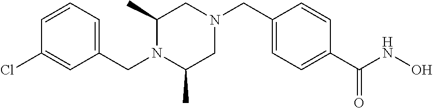 [SMILES: C[C@@H]1CN(Cc2ccc(C(=O)NO)cc2)C[C@H](C)N1Cc1cccc(Cl)c1] |
| 35 | | 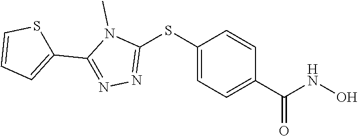 [SMILES: Cn1c(Sc2ccc(C(=O)NO)cc2)nnc1-c1cccs1] |
| 36 | | 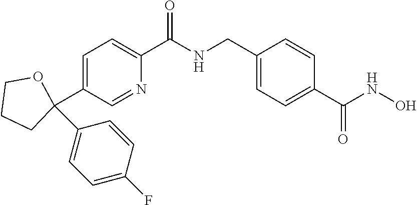 [SMILES: O=C(NO)c1ccc(CNC(=O)c2ccc(C3(c4ccc(F)cc4)CCCO3)cn2)cc1] |
| 37 | | 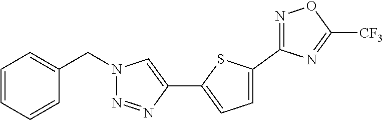 [SMILES: FC(F)(F)c1nc(-c2ccc(-c3cn(Cc4ccccc4)nn3)s2)no1] |
| 38 | | 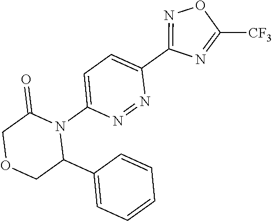 [SMILES: O=C1COCC(c2ccccc2)N1c1ccc(-c2noc(C(F)(F)F)n2)nn1] |
| 39 | | 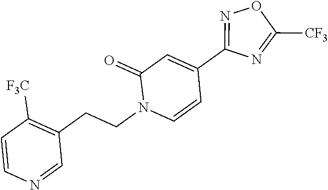 [SMILES: O=c1cc(-c2noc(C(F)(F)F)n2)ccn1CCc1cnccc1C(F)(F)F] |
| 40 | | 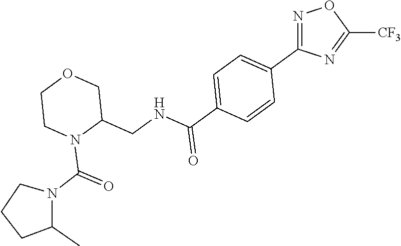 [SMILES: CC1CCCN1C(=O)N1CCOCC1CNC(=O)c1ccc(-c2noc(C(F)(F)F)n2)cc1] |
| 41 | | 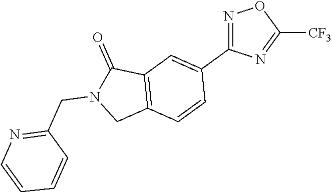 [SMILES: O=C1c2cc(-c3noc(C(F)(F)F)n3)ccc2CN1Cc1ccccn1] |
| 42 | | 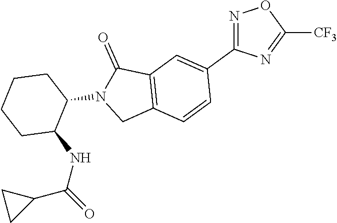 [SMILES: O=C(N[C@H]1CCCC[C@@H]1N1Cc2ccc(-c3noc(C(F)(F)F)n3)cc2C1=O)C1CC1] |
| 43 | | 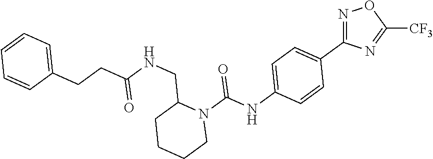 [SMILES: O=C(CCc1ccccc1)NCC1CCCCN1C(=O)Nc1ccc(-c2noc(C(F)(F)F)n2)cc1] |
| 44 | | 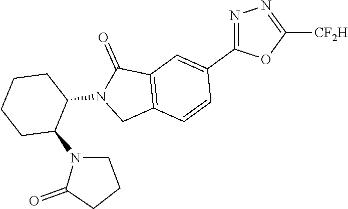 [SMILES: O=C1CCCN1[C@H]1CCCC[C@@H]1N1Cc2ccc(-c3nnc(C(F)F)o3)cc2C1=O] |
| 45 | | 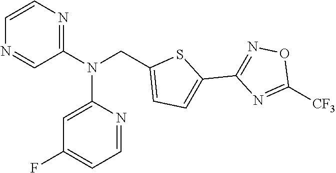 [SMILES: Fc1ccnc(N(Cc2ccc(-c3noc(C(F)(F)F)n3)s2)c2cnccn2)c1] |
| 46 | | 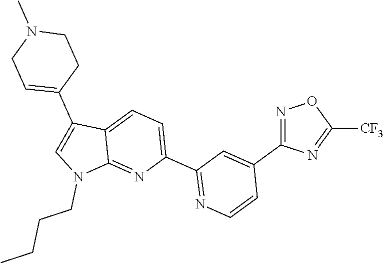 [SMILES: CCCCn1cc(C2=CCN(C)CC2)c2ccc(-c3cc(-c4noc(C(F)(F)F)n4)ccn3)nc21] |
| 47 | | 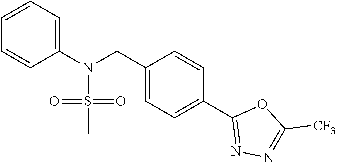 [SMILES: CS(=O)(=O)N(Cc1ccc(-c2nnc(C(F)(F)F)o2)cc1)c1ccccc1] |
| 48 | | 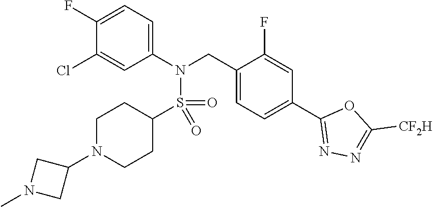 [SMILES: CN1CC(N2CCC(S(=O)(=O)N(Cc3ccc(-c4nnc(C(F)F)o4)cc3F)c3ccc(F)c(Cl)c3)CC2)C1] |
| 49 | | 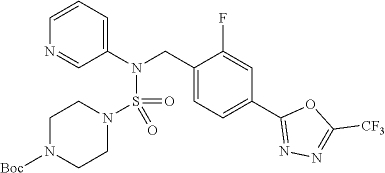 [SMILES: CC(C)(C)OC(=O)N1CCN(S(=O)(=O)N(Cc2ccc(-c3nnc(C(F)(F)F)o3)cc2F)c2cccnc2)CC1] |
| 50 | | 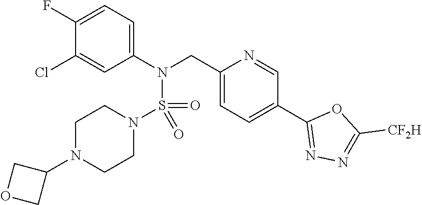 [SMILES: O=S(=O)(N1CCN(C2COC2)CC1)N(Cc1ccc(-c2nnc(C(F)F)o2)cn1)c1ccc(F)c(Cl)c1] |
| 51 | | 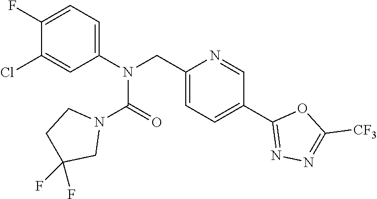 [SMILES: O=C(N1CCC(F)(F)C1)N(Cc1ccc(-c2nnc(C(F)(F)F)o2)cn1)c1ccc(F)c(Cl)c1] |
| 52 | | 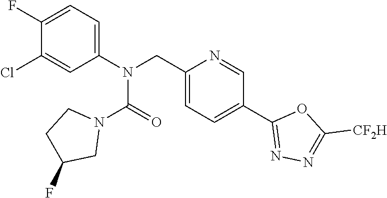 [SMILES: O=C(N1CC[C@H](F)C1)N(Cc1ccc(-c2nnc(C(F)F)o2)cn1)c1ccc(F)c(Cl)c1] |
| 53 | | 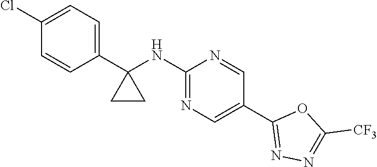 [SMILES: FC(F)(F)c1nnc(-c2cnc(NC3(c4ccc(Cl)cc4)CC3)nc2)o1] |
| 54 | | 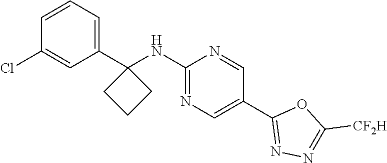 [SMILES: FC(F)c1nnc(-c2cnc(NC3(c4cccc(Cl)c4)CCC3)nc2)o1] |
| 55 | SS-1-100 | 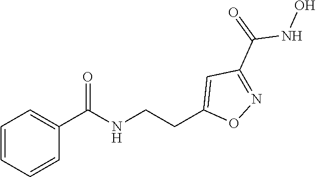 [SMILES: O=C(NCCc1cc(C(=O)NO)no1)c1ccccc1] |
| 56 | SS-2-08 | 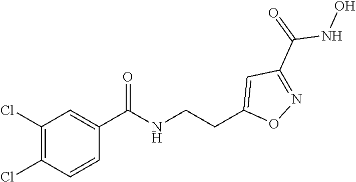 [SMILES: O=C(NCCc1cc(C(=O)NO)no1)c1ccc(Cl)c(Cl)c1] |
| 57 | SS-3-66 | 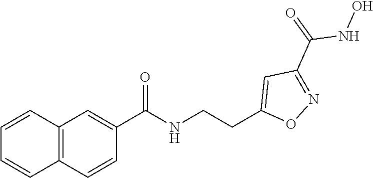 [SMILES: O=C(NCCc1cc(C(=O)NO)no1)c1ccc2ccccc2c1] |
| 58 | SS-3-67 | 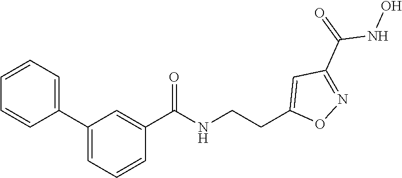 [SMILES: O=C(NCCc1cc(C(=O)NO)no1)c1cccc(-c2ccccc2)c1] |
| 59 | SS-3-94 | 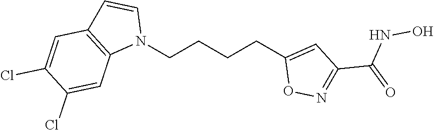 [SMILES: O=C(NO)c1cc(CCCCn2ccc3cc(Cl)c(Cl)cc32)on1] |
| 60 | SS-4-01 | 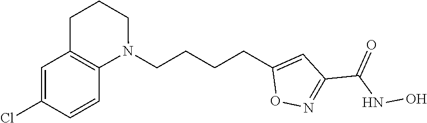 [SMILES: O=C(NO)c1cc(CCCCN2CCCc3cc(Cl)ccc32)on1] |
| 61 | SS-4-02 | 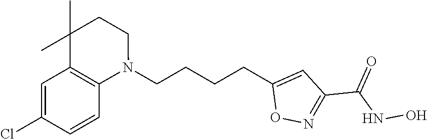 [SMILES: CC1(C)CCN(CCCCc2cc(C(=O)NO)no2)c2ccc(Cl)cc21] |
| 62 | SS-4-10 | 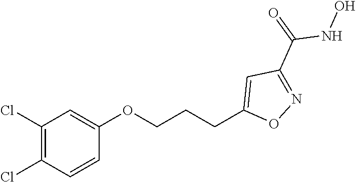 [SMILES: O=C(NO)c1cc(CCCOc2ccc(Cl)c(Cl)c2)on1] |
| 63 | SS-1-54 | 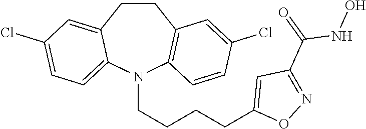 [SMILES: O=C(NO)c1cc(CCCCN2c3ccc(Cl)cc3CCc3cc(Cl)ccc32)on1] |
| 64 | | 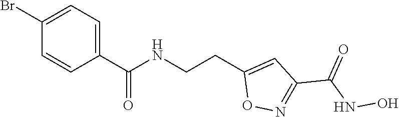 [SMILES: O=C(NCCc1cc(C(=O)NO)no1)c1ccc(Br)cc1] |
| 65 | | 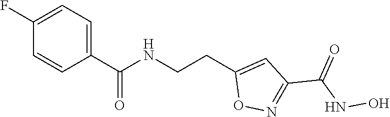 [SMILES: O=C(NCCc1cc(C(=O)NO)no1)c1ccc(F)cc1] |
| 66 | | 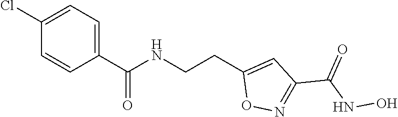 [SMILES: O=C(NCCc1cc(C(=O)NO)no1)c1ccc(Cl)cc1] |
| 67 | | 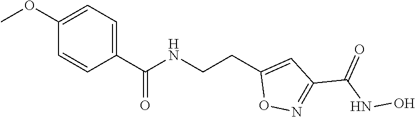 [SMILES: COc1ccc(C(=O)NCCc2cc(C(=O)NO)no2)cc1] |
| 68 | | 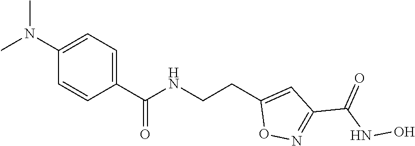 [SMILES: CN(C)c1ccc(C(=O)NCCc2cc(C(=O)NO)no2)cc1] |
| 69 | | 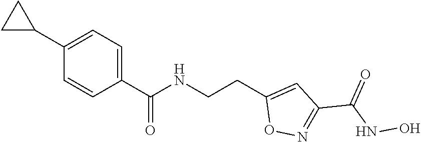 [SMILES: O=C(NCCc1cc(C(=O)NO)no1)c1ccc(C2CC2)cc1] |
| 70 | | 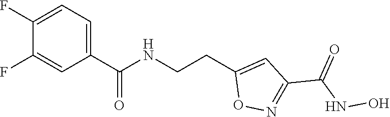 [SMILES: O=C(NCCc1cc(C(=O)NO)no1)c1ccc(F)c(F)c1] |
| 71 | | 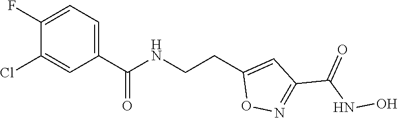 [SMILES: O=C(NCCc1cc(C(=O)NO)no1)c1ccc(F)c(Cl)c1] |
| 72 | | 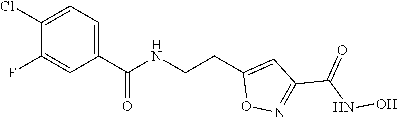 [SMILES: O=C(NCCc1cc(C(=O)NO)no1)c1ccc(Cl)c(F)c1] |
| 73 | | 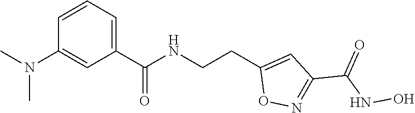 [SMILES: CN(C)c1cccc(C(=O)NCCc2cc(C(=O)NO)no2)c1] |
| 74 | | 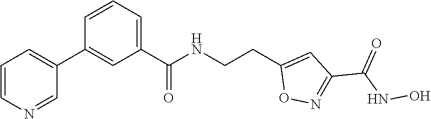 [SMILES: O=C(NCCc1cc(C(=O)NO)no1)c1cccc(-c2cccnc2)c1] |
| 75 | | 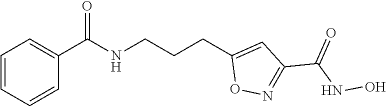 [SMILES: O=C(NCCCc1cc(C(=O)NO)no1)c1ccccc1] |
| 76 | | 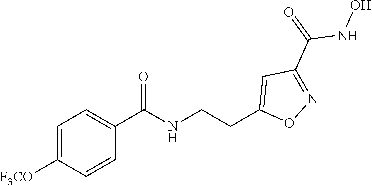 [SMILES: O=C(NCCc1cc(C(=O)NO)no1)c1ccc(OC(F)(F)F)cc1] |
| 77 | | 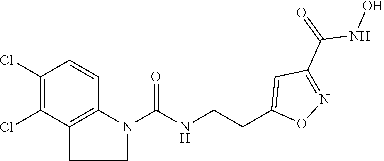 [SMILES: O=C(NO)c1cc(CCNC(=O)N2CCc3c2ccc(Cl)c3Cl)on1] |
| 78 | | 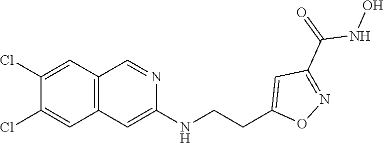 [SMILES: O=C(NO)c1cc(CCNc2cc3cc(Cl)c(Cl)cc3cn2)on1] |
| 79 | | 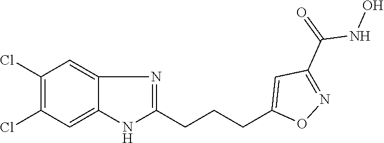 [SMILES: O=C(NO)c1cc(CCCc2nc3cc(Cl)c(Cl)cc3[nH]2)on1] |
| 80 | | 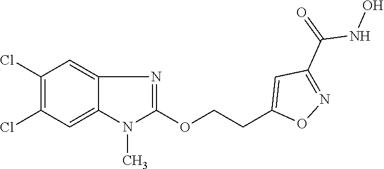 [SMILES: Cn1c(OCCc2cc(C(=O)NO)no2)nc2cc(Cl)c(Cl)cc21] |
| 81 | | 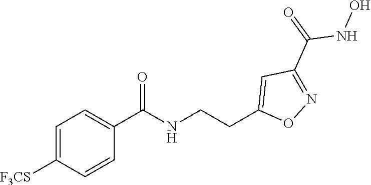 |
| 82 | | 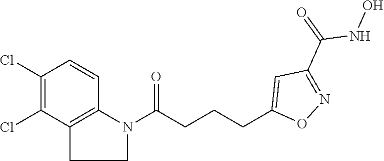 [SMILES: O=C(NO)c1cc(CCCC(=O)N2CCc3c2ccc(Cl)c3Cl)on1] |
| 83 | | 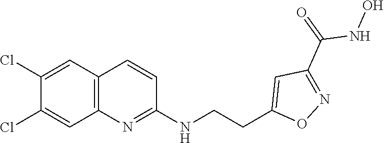 [SMILES: O=C(NO)c1cc(CCNc2ccc3cc(Cl)c(Cl)cc3n2)on1] |
| 84 | | 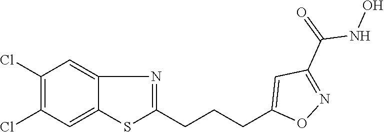 [SMILES: O=C(NO)c1cc(CCCc2nc3cc(Cl)c(Cl)cc3s2)on1] |
| 85 | | 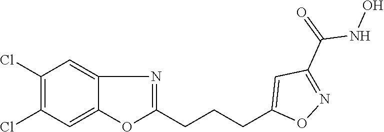 [SMILES: O=C(NO)c1cc(CCCc2nc3cc(Cl)c(Cl)cc3o2)on1] |
| 86 | | 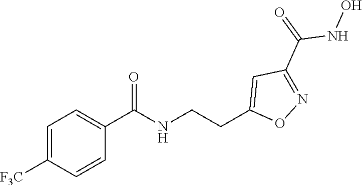 [SMILES: O=C(NCCc1cc(C(=O)NO)no1)c1ccc(C(F)(F)F)cc1] |
| 87 | | 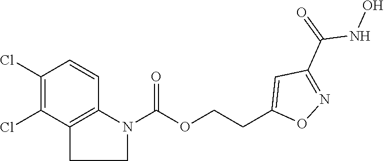 [SMILES: O=C(NO)c1cc(CCOC(=O)N2CCc3c2ccc(Cl)c3Cl)on1] |
| 88 | | 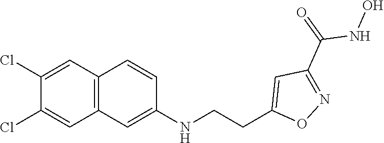 [SMILES: O=C(NO)c1cc(CCNc2ccc3cc(Cl)c(Cl)cc3c2)on1] |
| 89 | | 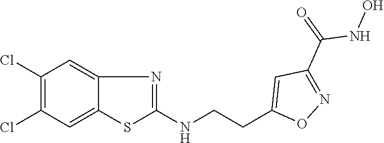 [SMILES: O=C(NO)c1cc(CCNc2nc3cc(Cl)c(Cl)cc3s2)on1] |
| 90 | | 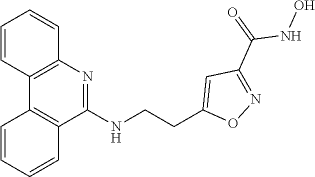 [SMILES: O=C(NO)c1cc(CCNc2nc3ccccc3c3ccccc23)on1] |
| 91 | | 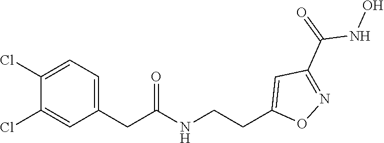 [SMILES: O=C(Cc1ccc(Cl)c(Cl)c1)NCCc1cc(C(=O)NO)no1] |
| 92 | | 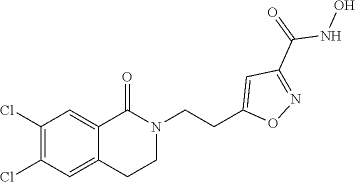 [SMILES: O=C(NO)c1cc(CCN2CCc3cc(Cl)c(Cl)cc3C2=O)on1] |
| 93 | | 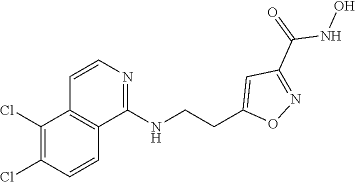 [SMILES: O=C(NO)c1cc(CCNc2nccc3c(Cl)c(Cl)ccc23)on1] |
| 94 | | 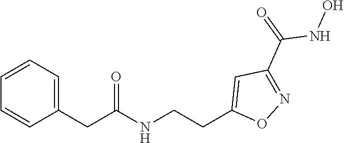 [SMILES: O=C(Cc1ccccc1)NCCc1cc(C(=O)NO)no1] |
| 95 | | 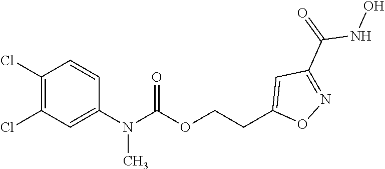 [SMILES: CN(C(=O)OCCc1cc(C(=O)NO)no1)c1ccc(Cl)c(Cl)c1] |
| 96 | | 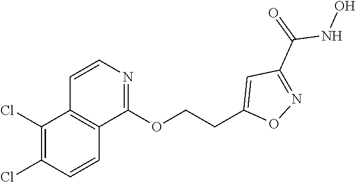 [SMILES: O=C(NO)c1cc(CCOc2nccc3c(Cl)c(Cl)ccc23)on1] |
| 97 | | 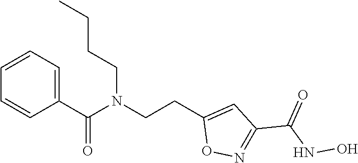 [SMILES: CCCCN(CCc1cc(C(=O)NO)no1)C(=O)c1ccccc1] |
| 98 | | 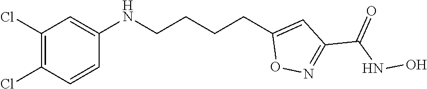 [SMILES: O=C(NO)c1cc(CCCCNc2ccc(Cl)c(Cl)c2)on1] |
| 99 | | 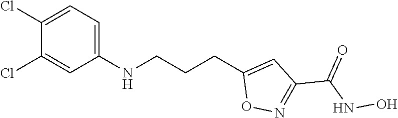 [SMILES: O=C(NO)c1cc(CCCNc2ccc(Cl)c(Cl)c2)on1] |
| 100 | | 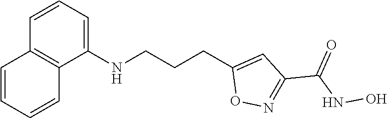 [SMILES: O=C(NO)c1cc(CCCNc2cccc3ccccc23)on1] |
| 101 | | 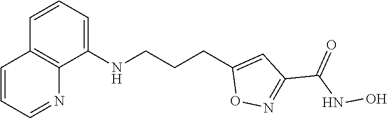 [SMILES: O=C(NO)c1cc(CCCNc2cccc3cccnc23)on1] |
| 102 | | 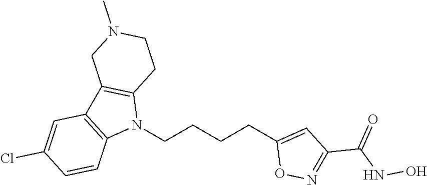 [SMILES: CN1CCc2c(c3cc(Cl)ccc3n2CCCCc2cc(C(=O)NO)no2)C1] |
| 103 | | 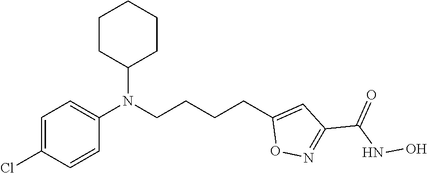 [SMILES: O=C(NO)c1cc(CCCCN(c2ccc(Cl)cc2)C2CCCCC2)on1] |
| 104 | | 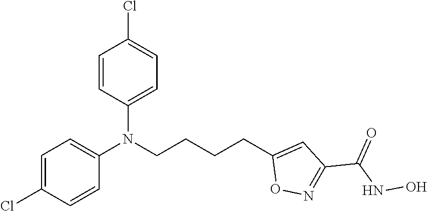 [SMILES: O=C(NO)c1cc(CCCCN(c2ccc(Cl)cc2)c2ccc(Cl)cc2)on1] |
| 105 | | 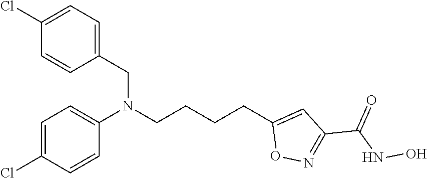 [SMILES: O=C(NO)c1cc(CCCCN(Cc2ccc(Cl)cc2)c2ccc(Cl)cc2)on1] |
| 106 | | 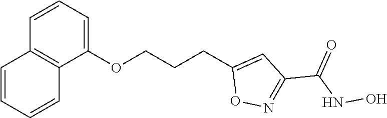 [SMILES: O=C(NO)c1cc(CCCOc2cccc3ccccc23)on1] |
| 107 | | 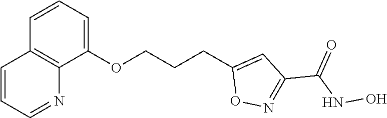 [SMILES: O=C(NO)c1cc(CCCOc2cccc3cccnc23)on1] |
| 108 | Tubacin | 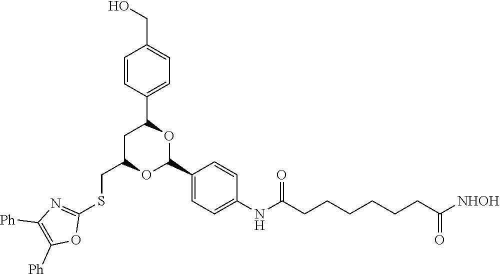 |
| 109 | | 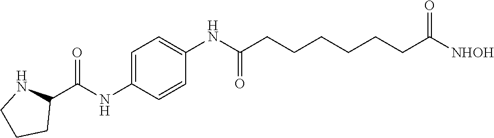 |
| 110 | | 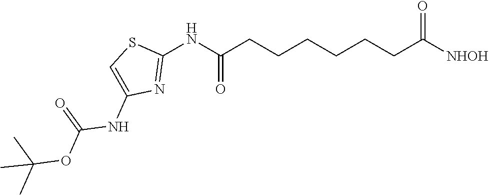 |
| 111 | | 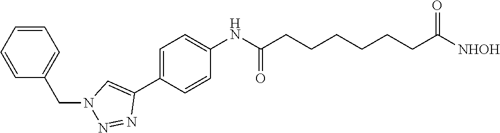 |
| 112 | | 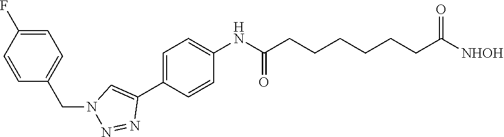 |
| 113 | CAY10603 | 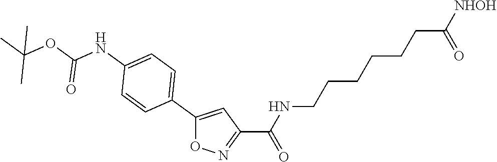 |
| 114 | | 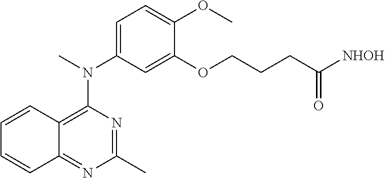 |
| 115 | | 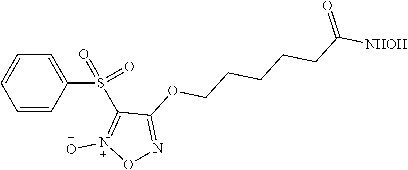 |
| 116 | | 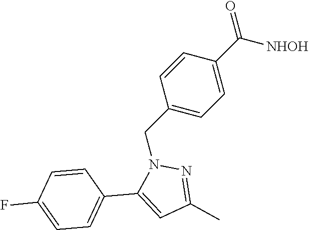 |
| 117 | | 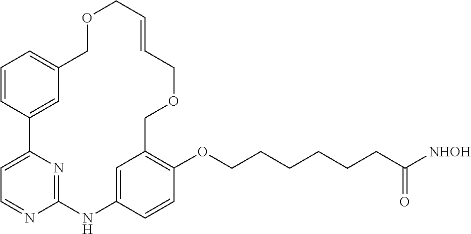 |
| 118 | | 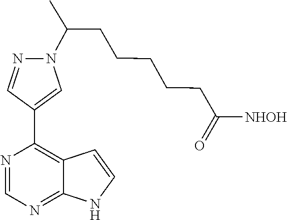 |
| 119 | | 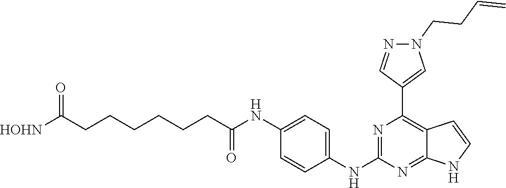 |
| 120 | | 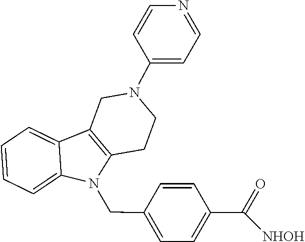 |
| 121 | | 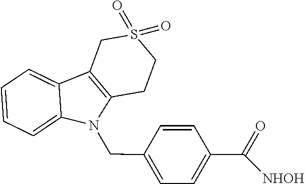 |
| 122 | | 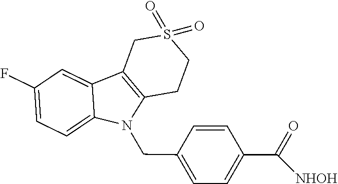 |
| 123 | | 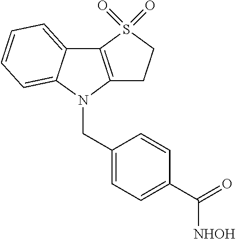 |
| 124 | | 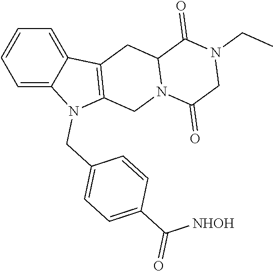 |
| 125 | | 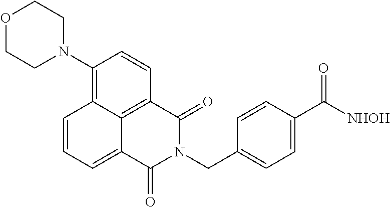 |
| 126 | | 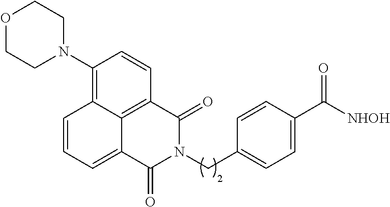 |
| 127 | | 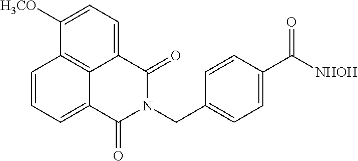 |
| 128 | | 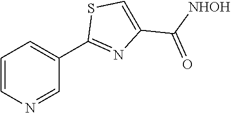 |
| 129 | | 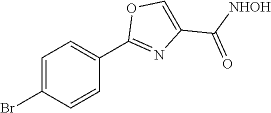 |
| 130 | | 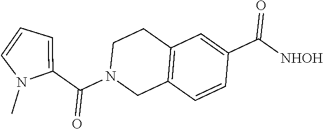 |
| 131 | | 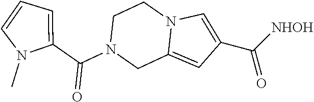 |
| 132 | | 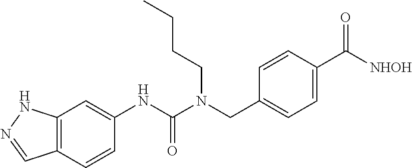 |
| 133 | | 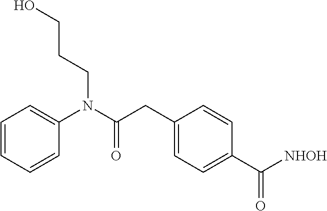 |
| 134 | | 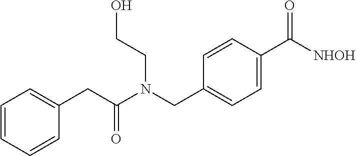 |
| 135 | | 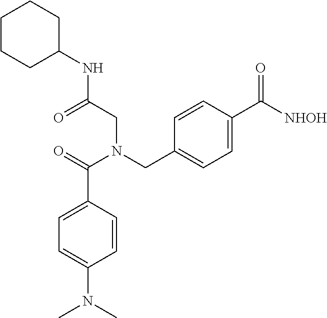 |
| 136 | | 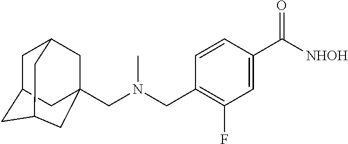 |
| 137 | | 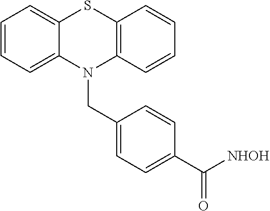 |
| 138 | | 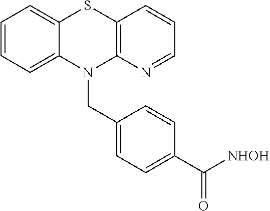 |
| 139 | | 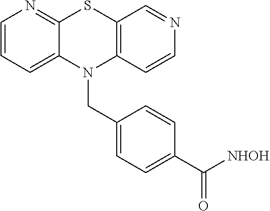 |
| 140 | | 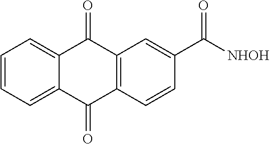 |
| 141 | HPOB | 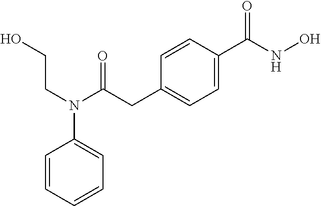 [SMILES: O=C(NO)c1ccc(CC(=O)N(CCO)c2ccccc2)cc1] |
| 142 | MPT0G413 | 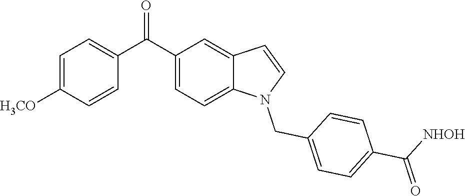 |
| 143 | CKD-506 | |
| 144 | WT161 | 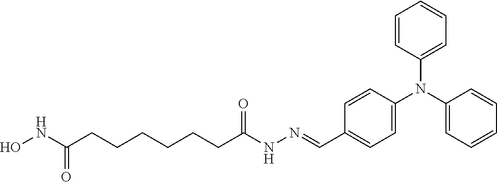 [SMILES: O=C(CCCCCCC(=O)N/N=C/c1ccc(N(c2ccccc2)c2ccccc2)cc1)NO] |
| 145 | KA2507 | 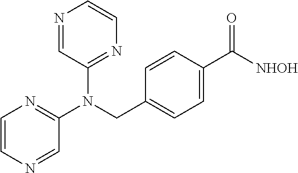 |

In the present disclosure, the term “macrophage polarizing agent” as used herein refers to an agent that polarizes a macrophage. Macrophage polarization is a process by which macrophages adopt different functional programs in response to the signals from their microenvironment. The polarization of macrophages can give a diverse heterogenic function and phenotypes depending on their activation in respect to their duration of stimulation and spatial localization. Non-limiting exemplary macrophage polarizing agent include, but are not limited to, lipopolysaccharide (LPS), interferon-gamma (IFN-γ), interleukin-2, interleukin-3, interleukin-4, interleukin-5, interleukin-6, interleukin-10, interleukin-12, interleukin-13, interleukin-18, interleukin-23, transforming growth factor beta (TGF-β), glucocorticoids, lipoteichoic acid (LTA), granulocyte-macrophage colony-stimulating factor (GM-CSF), tumor necrosis factor (TNF), immune complexes (IC), interleukin-1β, adenosines, or the combination thereof. See, e.g., Rubio et al., Clinical and Translational Oncology 21:391-403 (2019).

In the present disclosure, the term “tumor antigen” as used herein refers to an antigenic substance that can be produced in tumor cells and trigger an immune response in the host. Tumor antigens can be classified into two categories. One category is products of mutated oncogenes and tumor suppressor genes, and the other category is products of other mutated genes which include overexpressed or aberrantly expressed cellular proteins, tumor antigens produced by oncogenic viruses, oncofetal antigens, altered cell surface glycolipids and glycoproteins, and cell type-specific differentiation antigens. Non-limiting exemplary tumor antigens include, but are not limited to, Alphafetoprotein (AFP), Carcinoembryonic antigen (CEA), cancer antigen 125 (CA-125), cancer antigen 15-3 (CA 15-3), cancer antigen 19-9 (CA 19-9), mucin 1 (MUC-1), Epithelial tumor antigen (ETA), Tyrosinase, Melanoma-associated antigen (MAGE), B melanoma antigen (BAGE), CAGE, New York esophageal squamous cell carcinoma 1 (NY-ESO-1), melanoma antigen recognized by T cells 1 (MART-1), prostate specific antigen (PSA), human epidermal growth factor receptor 2 (HER2), calcitonin, and p53. In some embodiments, the one or more tumor antigens comprise alphafetoprotein (AFP), carcinoembryonic antigen (CEA), CA-125, MUC-1, epithelial tumor antigen (ETA), tyrosinase, Melanoma-associated antigen (MAGE), or p53, or a combination thereof.

In the present disclosure, the term “halo” or “halogen” as used by itself or as part of another group refers to —Cl, —F, —Br, or —I. In one aspect, the halo is —Cl or —F. In one aspect, the halo is —Cl.

In the present disclosure, the term “nitro” as used by itself or as part of another group refers to —NO₂.

In the present disclosure, the term “cyano” as used by itself or as part of another group refers to —CN.

In the present disclosure, the term “hydroxy” as used by itself or as part of another group refers to —OH.

In the present disclosure, the term “alkyl” as used by itself or as part of another group refers to unsubstituted straight- or branched-chain aliphatic hydrocarbons containing from one to twelve carbon atoms, i.e., C₁₋₁₂ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one aspect, the alkyl is a C₁₋₁₀ alkyl. In another aspect, the alkyl is a C₁₋₆ alkyl. In another aspect, the alkyl is a C₁₋₄ alkyl. In another aspect, the alkyl is a straight chain C₁₋₁₀ alkyl. In another aspect, the alkyl is a branched chain C₃₋₁₀ alkyl. In another aspect, the alkyl is a straight chain C₁₋₆ alkyl. In another aspect, the alkyl is a branched chain C₃₋₆ alkyl. In another aspect, the alkyl is a straight chain C₁₋₄ alkyl. In another aspect, the alkyl is a branched chain C₃₋₄ alkyl. In another aspect, the alkyl is a straight or branched chain C₃₋₄ alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

In the present disclosure, the term “cycloalkyl” as used by itself or as part of another group refers to saturated and partially unsaturated (containing one or two double bonds) cyclic aliphatic hydrocarbons containing one to three rings having from three to twelve carbon atoms, i.e., C₃₋₁₂ cycloalkyl. or the number of carbons designated. In one aspect, the cycloalkyl group has two rings. In one aspect, the cycloalkyl group has one ring. In another aspect, the cycloalkyl group is chosen from a C₃₋₈ cycloalkyl group. In another aspect, the cycloalkyl group is chosen from a C₃₋₆ cycloalkyl group. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, cyclopentenyl, and cyclohexenyl.

In the present disclosure, the term “optionally substituted cycloalkyl” as used by itself or as part of another group means that the cycloalkyl as defined above is either unsubstituted or substituted with one, two, or three substituents independently selected from the group consisting of halogen, hydroxy, nitro, cyano, —SCH₃, —SCF₃, —NR^{a}R^{b}, —C(O)NR^{a}R^{b}, —C(═O)CH₃, C₁₋₆ alkyl, C₂₋₆ alkenyl, C₂₋₆ alkynyl, C₁₋₆ alkoxy, C₁₋₆ haloalkyl, optionally substituted C₃₋₈ cycloalkyl, optionally substituted aryl, optionally substituted heteroaryl, and optionally substituted heterocyclo. In one aspect, the optionally substituted cycloalkyl is substituted with two substituents. In another aspect, the optionally substituted cycloalkyl is substituted with one substituent.

In the present disclosure, the term “alkenyl” as used by itself or as part of another group refers to an alkyl group as defined above containing one, two or three carbon-to-carbon double bonds. In one aspect, the alkenyl group is chosen from a C₂₋₆ alkenyl group. In another aspect, the alkenyl group is chosen from a C₂₋₄ alkenyl group. Non-limiting exemplary alkenyl groups include ethenyl, propenyl, isopropenyl, butenyl, sec-butenyl, pentenyl, and hexenyl.

In the present disclosure, the term “alkynyl” as used by itself or as part of another group refers to an alkyl group as defined above containing one to three carbon-to-carbon triple bonds. In one aspect, the alkynyl has one carbon-to-carbon triple bond. In one aspect, the alkynyl group is chosen from a C₂₋₆ alkynyl group. In another aspect, the alkynyl group is chosen from a C₂₋₄ alkynyl group. Non-limiting exemplary alkynyl groups include ethynyl, propynyl, butynyl, 2-butynyl, pentynyl, and hexynyl groups.

In the present disclosure, the term “haloalkyl” as used by itself or as part of another group refers to an alkyl group substituted by one or more fluorine, chlorine, bromine and/or iodine atoms. In one aspect, the alkyl group is substituted by one, two, or three fluorine and/or chlorine atoms. In another aspect, the haloalkyl group is a C₁₋₆ haloalkyl group. In another aspect, the haloalkyl group is a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “alkoxy” as used by itself or as part of another group refers to an optionally substituted alkyl, optionally substituted cycloalkyl, optionally substituted alkenyl or optionally substituted alkynyl attached to a terminal oxygen atom. In one aspect, the alkoxy group is chosen from a C₁₋₄ alkoxy group. In another aspect, the alkoxy group is chosen from a C₁₋₆ alkoxy group. In another aspect, the alkoxy group is chosen from a C₁₋₄ alkyl attached to a terminal oxygen atom, e.g., methoxy, ethoxy, and tert-butoxy.

In the present disclosure, the term “haloalkoxy” as used by itself or as part of another group refers to a C₁₋₄ haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “aryl” as used by itself or as part of another group refers to a monocyclic, bicyclic, or tricyclic aromatic ring system having from six to fourteen carbon atoms, i.e., C₆-C₁₄ aryl. Non-limiting exemplary aryl groups include phenyl (abbreviated as “Ph”), 1-naphthyl, 1-naphthyl, phenanthryl, anthracyl, indenyl, azulenyl, biphenyl, biphenylenyl, and fluorenyl groups. In one aspect, the aryl group is chosen from phenyl, 1-naphthyl, or 2-naphthyl. In one aspect, the aryl is a bicyclic or tricyclic C₁₀-C₁₄ aromatic ring system.

In the present disclosure, the term “optionally substituted aryl” as used herein by itself or as part of another group means that the aryl as defined above is either unsubstituted or substituted with one to five substituents independently selected from the group consisting of halogen, hydroxy, nitro, cyano, —SCH₃, —SCF₃, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₁₂ cycloalkyl, optionally substituted C₆-C₁₄ aryl, optionally substituted 5- to 14-membered heteroaryl, and optionally substituted 3- to 14-membered heterocyclo, wherein R^{a} and R^{b} are independently selected from the group consisting of hydrogen and C₁₋₆ alkyl; or R^{a} and R^{b} taken together with the nitrogen atom to which they are attached form a 3- to 12-membered heterocyclo; and R^{c} is C₁₋₄ alkyl.

In one aspect, the optionally substituted aryl is an optionally substituted phenyl. In one aspect, the optionally substituted phenyl has four substituents. In another aspect, the optionally substituted phenyl has three substituents. In another aspect, the optionally substituted phenyl has two substituents. In another aspect, the optionally substituted phenyl has one substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl, 3,4-di-chlorophenyl, 3,5-di-methylphenyl, 3,5-dimethoxy, 4-methylphenyl, 2-fluoro-3-chlorophenyl, and 3-chloro-4-fluorophenyl. The term optionally substituted aryl is meant to include groups having fused optionally substituted cycloalkyl and fused optionally substituted heterocyclo rings. Non-limiting examples include:

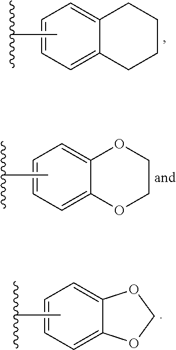

In the present disclosure, the term “heteroaryl” refers to monocyclic, bicyclic, and tricyclic aromatic ring systems having 5 to 14 ring atoms, i.e., a 5- to 14-membered heteroaryl, wherein at least one carbon atom of one of the rings is replaced with a heteroatom independently selected from the group consisting of oxygen, nitrogen and sulfur. In one aspect, the heteroaryl contains 1, 2, 3, or 4 heteroatoms independently selected from the group consisting of oxygen, nitrogen and sulfur. In one aspect, the heteroaryl has three heteroatoms. In another aspect, the heteroaryl has two heteroatoms. In another aspect, the heteroaryl has one heteroatom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one aspect, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl), isoxazolyl (e.g., isoxazol-3-yl, isoxazol-4-yl, and isoxazol-5-yl), and indazolyl (e.g., 1H-indazol-3-yl). The term “heteroaryl” is also meant to include possible N-oxides. A non-limiting exemplary N-oxide is pyridyl N-oxide.

In one aspect, the heteroaryl is a 5- or 6-membered heteroaryl. In one aspect, the heteroaryl is a 5-membered heteroaryl, i.e., the heteroaryl is a monocyclic aromatic ring system having 5 ring atoms wherein at least one carbon atom of the ring is replaced with a heteroatom independently selected from nitrogen, oxygen, and sulfur. Non-limiting exemplary 5-membered heteroaryl groups include thienyl, furyl, pyrrolyl, oxazolyl, pyrazolyl, imidazolyl, thiazolyl, isothiazolyl, and isoxazolyl.

In another aspect, the heteroaryl is a 6-membered heteroaryl, e.g., the heteroaryl is a monocyclic aromatic ring system having 6 ring atoms wherein at least one carbon atom of the ring is replaced with a nitrogen atom. Non-limiting exemplary 6 membered heteroaryl groups include pyridyl, pyrazinyl, pyrimidinyl, and pyridazinyl.

In another aspect, the heteroaryl is a 9- to 14-membered bicyclic aromatic ring system, wherein at least one carbon atom of one of the rings is replaced with a heteroatom independently selected from the group consisting of oxygen, nitrogen and sulfur. Non-limiting exemplary 9- to 14-membered bicyclic aromatic ring systems include:

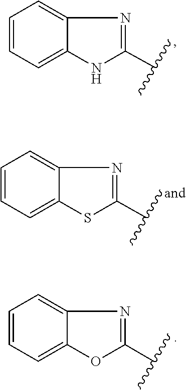 [SMILES: *c1nc2ccccc2[nH]1 ; *c1nc2ccccc2s1 ; *c1nc2ccccc2o1]

In the present disclosure, the term “optionally substituted heteroaryl” as used by itself or as part of another group means that the heteroaryl as defined above is either unsubstituted or substituted with one to four substituents independently selected from the group consisting of halogen, hydroxy, nitro, cyano, —SCH₃, —SCF₃, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₁₂ cycloalkyl, optionally substituted C₆-C₁₄ aryl, optionally substituted 5- to 14-membered heteroaryl, and optionally substituted 3- to 14-membered heterocyclo, wherein R^{a} and R^{b} are independently selected from the group consisting of hydrogen and C₁₋₆ alkyl; or R^{a} and R^{b} taken together with the nitrogen atom to which they are attached form a 3- to 12-membered heterocyclo; and R^{c} is C₁₋₄ alkyl. In one aspect, the optionally substituted heteroaryl has one substituent. Any available carbon or nitrogen atom can be substituted.

In the present disclosure, the term “heterocycle” or “heterocyclo” as used by itself or as part of another group refers to saturated and partially unsaturated, e.g., containing one or two double bonds, cyclic groups containing one, two, or three rings having from three to fourteen ring members, i.e., a 3- to 14-membered heterocyclo, wherein at least one carbon atom of one of the rings is replaced with a heteroatom. Each heteroatom is independently selected from the group consisting of oxygen, sulfur, including sulfoxide and sulfone, and/or nitrogen atoms, which can be oxidized or quaternized. The term “heterocyclo” is meant to include groups wherein a ring —CH₂— is replaced with a —C(═O)—, for example, cyclic ureido groups such as 2-imidazolidinone and cyclic amide groups such as β-lactam, γ-lactam, δ-lactam, ε-lactam, and piperazin-2-one. The term “heterocyclo” is also meant to include groups having fused optionally substituted aryl groups, e.g., indolinyl. In one aspect, the heterocyclo group is chosen from a 5- or 6-membered cyclic group containing one ring and one or two oxygen and/or nitrogen atoms. The heterocyclo can be optionally linked to the rest of the molecule through any available carbon or nitrogen atom. Non-limiting exemplary heterocyclo groups include dioxanyl, tetrahydropyranyl, 2-oxopyrrolidin-3-yl, piperazin-2-one, piperazine-2,6-dione, 2-imidazolidinone, piperidinyl, morpholinyl, piperazinyl, pyrrolidinyl, and indolinyl.

In the present disclosure, the term “optionally substituted heterocyclo” as used herein by itself or part of another group means the heterocyclo as defined above is either unsubstituted or substituted with one to four substituents independently selected from the group consisting of halogen, hydroxy, nitro, cyano, —SCH₃, —SCF₃, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₁₂ cycloalkyl, optionally substituted C₆-C₁₄ aryl, optionally substituted 5- to 14-membered heteroaryl, and optionally substituted 3- to 14-membered heterocyclo, wherein R^{a} and R^{b} are independently selected from the group consisting of hydrogen and C₁₋₆ alkyl; or R^{a} and R^{b} taken together with the nitrogen atom to which they are attached form a 3- to 12-membered heterocyclo; and R^{c} is C₁₋₄ alkyl.

In the present disclosure, the term “aralkyl” as used by itself or as part of another group refers to an alkyl group substituted with one, two, or three optionally substituted aryl groups. In one aspect, the optionally substituted aralkyl group is a C₁₋₄ alkyl substituted with one optionally substituted aryl group. In one aspect, the aralkyl group is a C₁ or C₂ alkyl substituted with one optionally substituted aryl group. In one aspect, the aralkyl group is a C₁ or C₂ alkyl substituted with one optionally substituted phenyl group. Non-limiting exemplary aralkyl groups include benzyl, phenethyl, —CHPh₂, —CH₂(4-F-Ph), —CH₂(4-Me-Ph), —CH₂(4-CF₃-Ph), and —CH(4-F-Ph)₂.

In the present disclosure, the term “heteroaralkyl” as used by itself or as part of another group refers to an alkyl group substituted with one, two, or three optionally substituted heteroaryl groups. In one aspect, the heteroaralkyl group is a C₁₋₄ alkyl substituted with one optionally substituted heteroaryl group. In one aspect, the aralkyl group is a C₁ or C₂ alkyl substituted with one optionally substituted heteroaryl group. In one aspect, the heteroaralkyl group is a C₁ or C₂ alkyl substituted with one optionally substituted heteroaryl group. Non-limiting exemplary heteroaralkyl groups include:

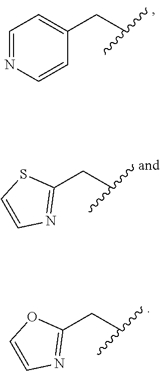 [SMILES: *Cc1ccncc1 ; *Cc1nccs1 ; *Cc1ncco1]

The term “HDAC” refers to a family of enzymes that remove acetyl groups from a protein, for example, the ε-amino groups of lysine residues at the N-terminus of a histone. The HDAC can be any human HDAC isoform including, HDAC1, HDAC2, HDAC3, HDAC4, HDAC5, HDAC6, HDAC7, HDAC8, HDAC9, HDAC10, and HDAC11. The HDAC also can be derived from a protozoal or fungal source.

The terms “treat,” “treating,” “treatment,” and the like refer to eliminating, reducing, relieving, reversing, and/or ameliorating a disease or condition and/or symptoms associated therewith. Although not precluded, treating a disease or condition does not require that the disease, condition, or symptoms associated therewith be completely eliminated, including the treatment of acute or chronic signs, symptoms and/or malfunctions. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition, “treatment” therefore also includes relapse prophylaxis or phase prophylaxis. The term “treat” and synonyms contemplate administering a therapeutically effective amount of a compound of the disclosure to an individual, e.g., a mammalian patient including, but not limited to, humans and veterinary animals, in need of such treatment. A treatment can be orientated symptomatically, for example, to suppress symptoms. It can be effected over a short period, be oriented over a medium term, or can be a long-term treatment, for example within the context of a maintenance therapy.

The term “therapeutically effective amount” or “therapeutic dose” as used herein refers to an amount of the active ingredient(s) that, when administered, is (are) sufficient, to efficaciously deliver the active ingredient(s) for the treatment of condition or disease of interest to an individual, e.g., human patient, in need thereof. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; reduce HDAC signaling in the target cells; and/or relieve, to some extent, one or more of the symptoms associated with the cancer. To extent the administered compound or composition prevents growth and/or kills existing cancer cells, it may be cytostatic and/or cytotoxic.

The use of the terms “a”, “an”, “the”, and similar referents in the context of describing the disclosure (especially in the context of the claims) are to be construed to cover both the singular and the plural, unless otherwise indicated. Recitation of ranges of values herein merely serve as a shorthand method of referring individually to each separate value falling within the range, unless otherwise indicated herein, and each separate value and subrange is incorporated into the specification as if it were individually recited herein. The use of any and all examples, or exemplary language (e.g., “such as” and “like”) provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

The term “subject” as used herein refers to any human or mammal that is in need of or might benefit from treatment with STAT-activated macrophages. Foremost among such subjects are humans, although the methods and compositions provided herein are not intended to be so limited. Other subjects include veterinary animals, e.g., cows, sheep, pigs, horses, dogs, cats and the like. In one embodiment, the subject is a human. In one embodiment, the subject is a mammal.

Selective HDAC6 inhibitors can exist as salts. As used herein, the term “pharmaceutically acceptable salt” refers to salts or zwitterionic forms of the present compounds. Salts of the present compounds can be prepared during the final isolation and purification of the compounds or separately by reacting the compound with an acid having a suitable cation. The pharmaceutically acceptable salts of the present compounds can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, tartaric, and citric. Nonlimiting examples of salts of selective HDAC6 inhibitors include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in selective HDAC6 inhibitors can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides. Any reference to compounds of the present disclosure appearing herein is intended to include selective HDAC6 inhibitors as well as pharmaceutically acceptable salts, solvates, or hydrates thereof.

### EXAMPLES

### General Methods

Bone marrow derived macrophages (BMDMs): For macrophage isolation, bone marrow from 6-12 weeks old C57BL/6 mouse was used following an IACUC approved protocol. Briefly, femurs and tibia bones were isolated after removing the skeletal muscles. The bone marrow was flushed with RPMI complete medium supplemented with non-essential amino acids. A single-cell suspension of bone marrow was prepared with repeated pipetting and incubated with 20 ng/mL of mouse recombinant M-CSF (Biolegend) at 37° C. for 4 days to differentiate into macrophages.

Flow cytometry: Flow cytometry was performed following the protocol described previously. Knox et al., Sci Rep. 2019 Oct. 10; 9(1):14824. doi: 10.1038/s41598-019-51403-6. Briefly, mice were euthanized following the IACUC protocol, and tumor cells were processed into a single cell suspension for analysis by flow cytometry with tumor digestion buffer. The following antibodies were used to stain cell surface markers expressed by different immune cells. All the antibodies were purchased from Biolegend (San Diego, CA) unless otherwise specified. Myeloid cell surface markers are as follows: APC anti-mouse CD80 (clone 16-10A1), PE/Cy7 anti-mouse CD206 (MMR) (clone C068C2), APC/Fire™ 750 anti-mouse CD45.2 (clone 104), FITC anti-mouse H-2 (clone M1/42), Brilliant Violet 785TM anti-mouse F4/80 (clone BM8), and Alexa Fluor® 700 anti-mouse CD3 (clone 17A2). To distinguish between MHCI and MHCI-bound to SIINFEKL, we used APC anti-mouse H-2Kb bound to SIINFEKL antibody (clone 25-D1.16). Multi-color flow data acquisition was performed on BD Celesta, and data analysis was performed with FlowJo software (version 10.3). Statistical analyses were performed with GraphPad Prism Software (version 7.03).

### Example 1

### Pretreatment of BMDMs with a STAT3 Inhibitor Decreases M2 Polarization

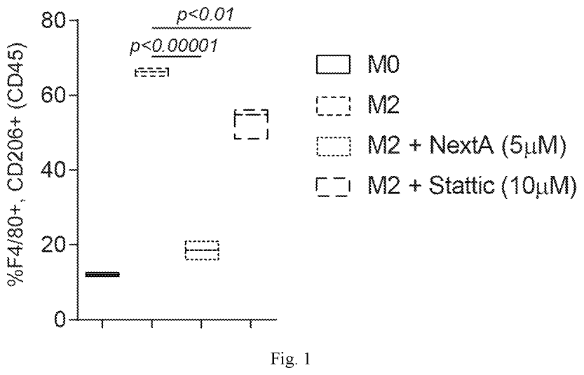

Bone marrow derived macrophages (BMDMs) were differentiated into macrophages with M-CSF. These naïve macrophages were pre-treated with Nexturastat A (NextA) or 6-nitrobenzo[b]thiophene-1,1-dioxide (Stattic) and polarized to M2 macrophages with IL4/IL13. Pre-treatment with NextA and Stattic decreased polarization of M2 macrophages as indicated by flow cytometry. See FIG. 1.

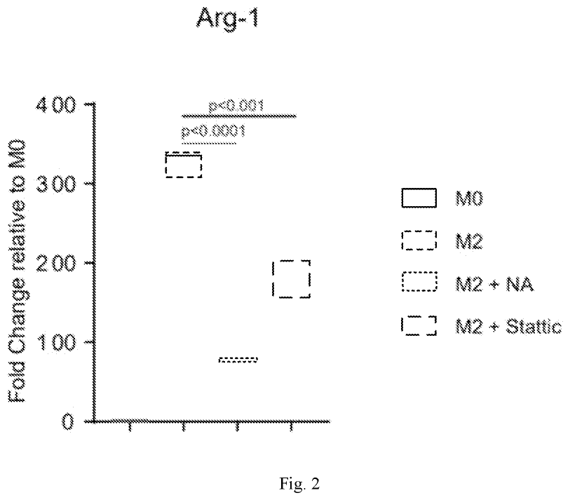

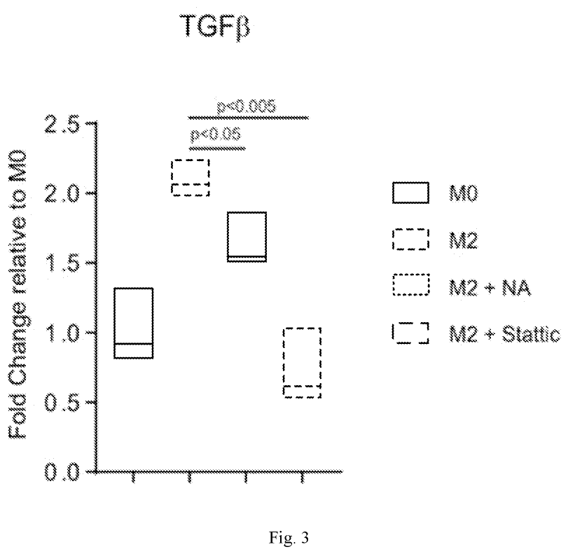

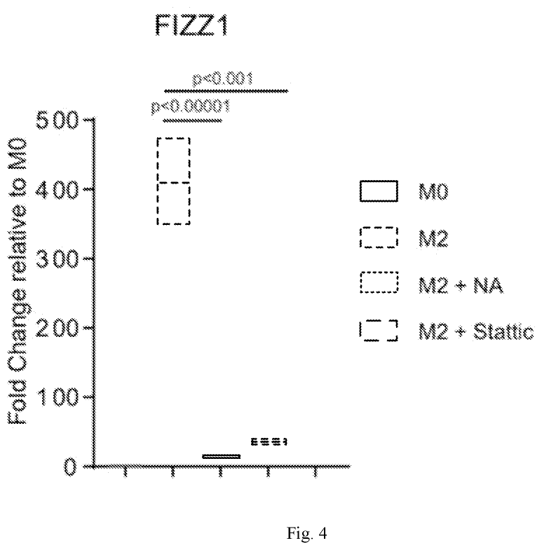

Further validation of M2 polarization markers at gene expression levels by quantitative real-time PCR indicate that Arginase I, TGFβ, and FIZZ1, which are tumor-promoting factors, were decreased. FIG. 2, FIG. 3, and FIG. 4. These data suggest that pre-treatment with NextA and Stattic affects cellular signaling and programming of naïve macrophages in response to polarizing factors in the tumor microenvironment (TME).

All of the features described herein (including any accompanying claims, abstract and drawings), and/or all of the steps of any method so disclosed, may be combined with any of the above aspects in any combination, except combinations where at least some of such features and/or steps are mutually exclusive.

It is to be understood that the foregoing aspects and exemplifications are not intended to be limiting in any respect to the scope of the disclosure, and that the claims presented herein are intended to encompass all aspects, embodiments, and exemplifications whether or not explicitly presented herein.

All patents, patent applications, and publications cited herein are fully incorporated by reference in their entirety.

What is claimed is:

1. Signal transducer and activator of transcription (STAT)-activated macrophages.

2. A composition comprising the STAT-activated macrophages of claim 1.

3. The composition of claim 2, wherein the STAT-activated macrophages are produced from naïve macrophages that have been isolated from a subject and treated ex vivo with a STAT inhibitor.

4. The composition of claim 3, wherein the subject is a mammal.

5. The composition of claim 4, wherein the subject is a human.

6. The composition of any one of claims 3-5, wherein the naïve macrophages are allogeneic macrophages, autologous macrophages, or a combination of allogeneic macrophages and autologous macrophages.

7. The composition of claim 6, wherein the naïve macrophages are autologous macrophages.

8. The composition of any one of claims 3-7, wherein the ex vivo treatment of the isolated naïve macrophages comprises one treatment with the STAT inhibitor.

9. The composition of any one of claims 3-7, wherein the ex vivo treatment of the isolated naïve macrophages comprises two or more treatments with the STAT inhibitor.

10. The composition of any one of claims 3-9, wherein the ex vivo treatment of the isolated naïve macrophages further comprises treatment with one or more macrophage polarizing agents.

11. The composition of claim 10, wherein the macrophage polarizing agent comprises lipopolysaccharide (LPS), interferon-gamma, interleukin-4 or interleukin-13, or a combination of thereof.

12. The composition of any one of claims 3-11, wherein the ex vivo treatment of the naïve macrophages further comprises treatment with one or more tumor antigens.

13. The composition of claim 12, wherein the one or more tumor antigens comprise alphafetoprotein (AFP), carcinoembryonic antigen (CEA), cancer antigen 125 (CA-125), cancer antigen 15-3 (CA 15-3), cancer antigen 19-9 (CA 19-9), mucin 1 (MUC-1), epithelial tumor antigen (ETA), tyrosinase, Melanoma-associated antigen (MAGE), B melanoma antigen (BAGE), CAGE, New York esophageal squamous cell carcinoma 1 (NY-ESO-1), melanoma antigen recognized by T cells 1 (MART-1), prostate specific antigen (PSA), human epidermal growth factor receptor 2 (HER2), calcitonin, or p53, or a combination thereof.

14. The composition of any one of claims 3-13, wherein the ex vivo treatment of the naïve macrophages further comprises treatment with one or more selective HDAC6 inhibitors.

15. The composition of claim 14, wherein the selective HDAC6 inhibitor is a compound of Formula I: 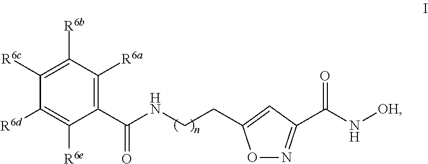
or a pharmaceutically acceptable salt thereof, wherein:
R^{6a}, R^{6b}, R^{6c}, R^{6d}, and R^{6e} are each independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₆ cycloalkyl, optionally substituted phenyl, optionally substituted 5- or 6-membered heteroaryl, and optionally substituted 5- or 6-membered heterocyclo;
R^{a} and R^{b} are independently selected from the group consisting of hydrogen and C₁₋₄ alkyl; or
R^{a} and R^{b} taken together with the nitrogen atom to which they are attached form a 3- to 10-membered heterocyclo;
R^{c} is C₁₋₄ alkyl; and
n is 1, 2, or 3.

16. The composition of claim 14, wherein the selective HDAC6 inhibitor is a compound of Formula II: 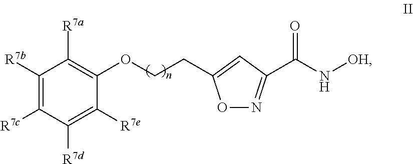
or a pharmaceutically acceptable salt thereof, wherein:
R^{7a}, R^{7b}, R^{7c}, R^{7d}, and R^{7e} are each independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₆ cycloalkyl, optionally substituted phenyl, optionally substituted 5- or 6-membered heteroaryl, and optionally substituted 5- or 6-membered heterocyclo;
R^{a} and R^{b} are independently selected from the group consisting of hydrogen and C₁₋₄ alkyl; or
R^{a} and R^{b} taken together with the nitrogen atom to which they are attached form a 3- to 10-membered heterocyclo;
R^{c} is C₁₋₄ alkyl; and
n is 1, 2, or 3.

17. The composition of claim 14, wherein the selective HDAC6 inhibitor is a compound of Formula III: 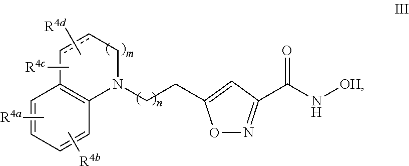
or a pharmaceutically acceptable salt thereof, wherein:
R^{4a} and R^{4b} are independently selected from the group consisting of hydrogen, halogen, cyano, C₁₋₄ alkyl, and C₁₋₄ alkoxy;
R^{4c} and R^{4d} are independently selected from the group consisting of hydrogen and methyl;
m is 0 or 1;
n is 1, 2, or 3; and [symbol]
represents a single or double bond.

18. The composition of claim 14, wherein the selective HDAC6 inhibitor is a compound of Formula IV: 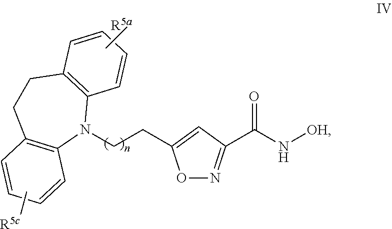
or a pharmaceutically acceptable salt thereof, wherein:
R^{5a} and R^{5c} are independently selected from the group consisting of hydrogen, halogen, cyano, C₁₋₄ alkyl, and C₁₋₄ alkoxy; and
n is 1, 2, or 3.

19. The composition of claim 14, wherein the selective HDAC6 inhibitor is a compound of Table 2, or a pharmaceutically acceptable salt thereof.

20. The composition of any one of claims 14-19, wherein the selective HDAC6 inhibitor is at least 20-fold selective over one or more other HDAC isoforms.

21. The composition of any one of claims 3-20, wherein the STAT inhibitor is a STAT3 inhibitor.

22. The composition of claim 21, wherein the STAT3 inhibitor is 6-nitrobenzo[b]thiophene-1,1-dioxide.

23. A method of producing STAT-activated macrophages, the method comprising isolating naïve macrophages from a subject and treating the isolated naïve macrophages ex vivo with a STAT inhibitor.

24. The method of claim 23, wherein the subject is a mammal.

25. The method of claim 24, wherein the subject is a human.

26. The method of any one of claims 23-25, wherein the naïve macrophages are allogeneic macrophages, autologous macrophages, or a combination of allogeneic macrophages and autologous macrophages.

27. The method of claim 26, wherein the naïve macrophages are autologous macrophages.

28. The method of any one of claims 23-27, wherein the ex vivo treatment of the naïve macrophages comprises one treatment with the STAT inhibitor.

29. The method of any one of claims 23-27, wherein the ex vivo treatment of the naïve macrophages comprises two or more treatments with the STAT inhibitor.

30. The method of any one of claims 23-29, wherein the ex vivo treatment of the naïve macrophages further comprises treatment with one or more macrophage polarizing agents.

31. The method of claim 30, wherein the macrophage polarizing agent comprises lipopolysaccharide (LPS), interferon-gamma, interleukin-4 or interleukin-13, or a combination of thereof.

32. The method of any one of claims 23-31, wherein the ex vivo treatment of the naïve macrophages further comprises treatment with one or more tumor antigens.

33. The method of claim 32, wherein the one or more tumor antigens comprise alphafetoprotein (AFP), carcinoembryonic antigen (CEA), cancer antigen 125 (CA-125), cancer antigen 15-3 (CA 15-3), cancer antigen 19-9 (CA 19-9), mucin 1 (MUC-1), epithelial tumor antigen (ETA), tyrosinase, Melanoma-associated antigen (MAGE), B melanoma antigen (BAGE), CAGE, New York esophageal squamous cell carcinoma 1 (NY-ESO-1), melanoma antigen recognized by T cells 1 (MART-1), prostate specific antigen (PSA), human epidermal growth factor receptor 2 (HER2), calcitonin, or p53, or a combination thereof.

34. The method of any one of claims 23-33, wherein the ex vivo treatment of the naïve macrophages further comprises treatment with one or more selective HDAC6 inhibitors.

35. The method of claim 34, wherein the selective HDAC6 inhibitor is a compound of Formula I: 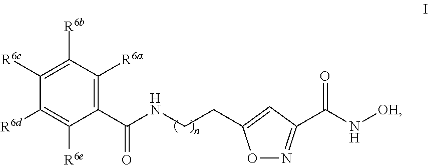
or a pharmaceutically acceptable salt thereof, wherein:
R^{6a}, R^{6b}, R^{6c}, R^{6d} and R^{6e} are each independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₆ cycloalkyl, optionally substituted phenyl, optionally substituted 5- or 6-membered heteroaryl, and optionally substituted 5- or 6-membered heterocyclo;
R^{a} and R^{b} are independently selected from the group consisting of hydrogen and C₁₋₄ alkyl; or
R^{a} and R^{b} taken together with the nitrogen atom to which they are attached form a 3-to 10-membered heterocyclo;
R^{c} is C₁₋₄ alkyl; and
n is 1, 2, or 3.

36. The method of claim 34, wherein the selective HDAC6 inhibitor is a compound of Formula II: 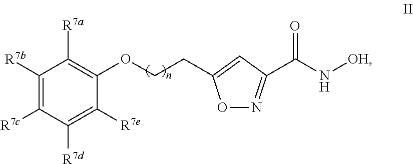
or a pharmaceutically acceptable salt thereof, wherein:
R^{7a}, R^{7b}, R^{7c}, R^{7d}, and R^{7e} are each independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, haloalkoxy, optionally substituted C₃₋₆ cycloalkyl, optionally substituted phenyl, optionally substituted 5- or 6-membered heteroaryl, and optionally substituted 5- or 6-membered heterocyclo;
R^{a} and R^{b} are independently selected from the group consisting of hydrogen and C₁₋₄ alkyl; or
R^{a} and R^{b} taken together with the nitrogen atom to which they are attached form a 3- to 10-membered heterocyclo;
R^{c} is C₁₋₄ alkyl; and
n is 1, 2, or 3.

37. The method of claim 34, wherein the selective HDAC6 inhibitor is a compound of Formula III: 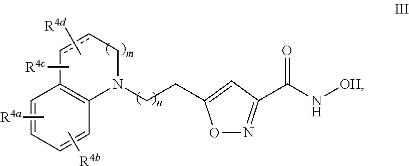
or a pharmaceutically acceptable salt thereof, wherein:
R^{4a} and R^{4b} are independently selected from the group consisting of hydrogen, halogen, cyano, C₁₋₄ alkyl, and C₁₋₄ alkoxy;
R^{4c} and R^{4d} are independently selected from the group consisting of hydrogen and methyl;
m is 0 or 1;
n is 1, 2, or 3; and [symbol]
represents a single or double bond.

38. The method of claim 34, wherein the selective HDAC6 inhibitor is a compound of Formula IV: 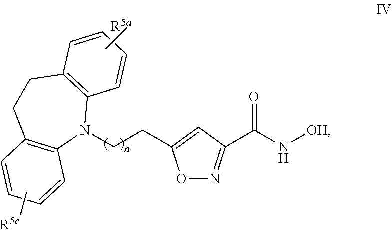
or a pharmaceutically acceptable salt thereof, wherein:
R^{5a} and R^{5c} are independently selected from the group consisting of hydrogen, halogen, cyano, C₁₋₄ alkyl, and C₁₋₄ alkoxy; and
n is 1, 2, or 3.

39. The method of claim 34, wherein the selective HDAC6 inhibitor is a compound of Table 2, or a pharmaceutically acceptable salt thereof.

40. The method of any one of claims 34-39, wherein the selective HDAC6 inhibitor inhibits HDAC6 at least 20-fold more than it inhibits one or more of HDAC1, HDAC2, HDAC3, HDAC4, HDAC5, HDAC7, HDAC8, HDAC9, HDAC10, or HDAC11.

41. The method of any one of claims 23-40, wherein the STAT inhibitor is a STAT3 inhibitor.

42. The method of claim 41, wherein the STAT3 inhibitor is 6-nitrobenzo[b]thiophene-1,1-dioxide.

43. STAT-activated macrophages obtained by the method of any one of claims 23-42.

44. A method of treating a subject in need thereof, the method comprising administering to the subject a therapeutically effective amount of the STAT-activated macrophages of claim 1, wherein the subject has cancer, pulmonary fibrosis, liver fibrosis, or heart fibrosis.

45. A method of treating a subject in need thereof, the method comprising administering to the subject a therapeutically effective amount of the composition of any one of claims 2-22, wherein the subject has cancer, pulmonary fibrosis, liver fibrosis, or heart fibrosis.

46. The method of claim 44 or 45, wherein the subject has cancer.

47. The method of claim 46 further comprising administering to the subject one or more of radiation therapy, immune checkpoint blockade therapy, photothermal therapy, or chemotherapy.

48. The method of claim 44 or 45, wherein the subject has pulmonary fibrosis.

49. The method of claim 44 or 45, wherein the subject has liver fibrosis.

50. The method of claim 44 or 45, wherein the subject has heart fibrosis.

51. The method of any one of claims 44-50, wherein the subject is a human.
